(19) United States
(12) Patent Application Publication
(10) Pub. No.: US 2005/0069985 A1
(43) Pub. Date: Mar. 31, 2005

(54) Assay methods and amelioration of muscular dystrophy symptoms

(75) Inventor: Stephen J. Kaufman, Urbana, IL (US)

Correspondence Address: GREENLEE WINNER AND SULLIVAN P C, 4875 PEARL EAST CIRCLE, SUITE 200, BOULDER, CO, 80301, US

(73) Assignee: The Board of Trustees of the University of Illinois, Urbana, IL (US)

(21) Appl. No.: 10/987,801

(22) Filed: Nov. 12, 2004

Related U.S. Application Data
(63) Continuation-in-part of application No. 10/081,885, filed on Feb. 20, 2002.
(60) Provisional application No. 60/270,645, filed on Feb. 20, 2001.
(60) Provisional application No. 60/286,890, filed on Apr. 27, 2001.

(51) Int. Cl. G01N 33/53; G01N 33/567; C07H 21/04; C12N 5/06; C12N 5/08

(52) U.S. Cl. 435/69.1; 435/368; 435/354; 435/320.1; 435/7.2; 530/350; 536/23.5

(57) ABSTRACT

The present disclosure provides methods for identifying compositions which increase the expression of α7 integrin protein in muscle cells of dystrophy patients. The present disclosure further provides compositions and sequences for the diagnosis, genetic therapy of certain muscular dystrophies, especially muscular dystrophy resulting from a deficiency in an α7 integrin protein or a dystrophin protein or a combined deficiency in dystrophin and utrophin, and methods and compositions for the identification of compounds which increase expression of the α7 integrin. Expression of the integrin αBX2 polypeptide in muscle cells results in better physical condition in a patient or an animal lacking normal levels of dystrophin or dystrophin and utrophin.

### CROSS-REFERENCE TO RELATED APPLICATIONS

This application is a Continuation-in-Part of U.S. patent application Ser. No. 10/081,885, filed Feb. 20, 2002, which application claims benefit of U.S. Provisional Application 60/270,645 filed Feb. 20, 2001, and from U.S. Provisional Application 60/286,890 filed Apr. 27, 2001, all of which are incorporated by reference herein.

### ACKNOWLEDGMENT OF FEDERAL RESEARCH SUPPORT

This invention was made, at least in part, with funding from the National Institutes of Health (Contract No. AG 14632). Accordingly, the United States government has certain rights in this invention.

### BACKGROUND OF THE INVENTION

The field of the present invention is the area of molecular technology, in particular, the present invention relates to assays for identifying compounds which induce increased expression via α7β1 integrin transcriptional regulatory sequences, especially as applied to drug induced gene expression to ameliorate the physical condition of muscular dystrophy patients, especially those lacking dystrophin or lacking dystrophin and utrophin or those with lower than normal levels of α7 integrin.

The defective association of skeletal and cardiac muscle with their surrounding basal lamina underlies the pathologies associated with a variety of muscular dystrophies and cardiomyopathies (Matsumura and Campbell, 1994; Hayashi, et al., 1993; Hayashi, et al., 1998; Lim and Campbell, 1998). Duchenne Muscular Dystrophy (DMD) is a congenital X-linked myopathy that is caused by a lack of the dystrophin protein and affects approximately 1 in 3300 males. Patients with DMD experience progressive muscle deterioration and debilitation that severely restricts mobility. Death due to cardiac and respiratory failure usually occurs in the second decade of life.

Mutations in the dystrophin gene result in a lack of dystrophin, a 427 kDa protein localized to the inner cytoplasmic side of the plasma membrane of skeletal and cardiac muscle cells (Monaco et a., 1986; Matsumura and Campbell, 1994; Campbell, 1995). In association with dystroglycans, syntrophins, and sarcoglycans, dystrophin links the cell cytoskeleton to laminin in the extracellular matrix. In the absence of one or more components of the dystrophin linkage system, the association of fibers with the surrounding basal lamina is compromised, leading to the myopathy observed. Thus, the molecular continuity between the extracellular matrix and the cell cytoskeleton is essential for the structural and functional integrity of muscle.

The integrins are a β heterodimeric receptors that bind extracellular matrix proteins and interact with the cell cytoskeleton (Hynes, 1992). The α7β1 integrin is a laminin receptor on skeletal and cardiac muscle (Song et al., 1992) and serves as a transmembrane link between the basal lamina and muscle fibers. Multiple isoforms of the α7 and β1 chains are generated by developmentally regulated RNA splicing resulting in a family of receptors with diverse structure and functions (for reviews see Hodges and Kaufman, 1996 and Burkin and Kaufman, 1999).

The α7 integrin chain is encoded by a single autosomal gene on human chromosome 12q13 (Wang et a., 1995). Three alternative cytoplasmic domain (α 7A, B and C) and two extracellular domain variants (X1 and X2) of the protein have been identified (Song, et al., 1993; Collo et al., 1993; Ziober et al., 1993). Four additional alternatively spliced isoforms of the extracellular domain have been predicted by nucleotide sequence analysis (Leung et al., 1998; Vignier, et al., 1999).

The α7β1 integrin is a major laminin receptor that serves as a transmembrane link and signal transduction mechanism between the extracellular matrix and the muscle fiber (Song et al. 1992; Hodges and Kaufman, 1996; Burkin and Kaufman, 1999). Alternative cytoplasmic domains (A, B and C) (Song et al. 1993; Collo et al., 1993; Zoiber et al., 1993) and extracellular domains (X1 and X2) (Zoiber et al., 1993, Hodges and Kaufman, 1996) of this integrin are generated by developmentally regulated alternative RNA splicing. The diversity in the α7 integrin chain appears to be the result of the broad range of biological functions with which it is associated during muscle development, including the development of neuromuscular junctions (Burkin et al., 1998; Burkin et al., 2000), stability of myotendinous junctions and overall muscle integrity (Hayashi et al., 1998).

The β1 chain cytoplasmic domain also undergoes developmentally regulated alternative splicing. βlA is the most common isoform of the β1 chain and is expressed in a wide variety of tissues including replicating myoblasts. The alternative β1D form is generated upon differentiation of myoblasts to myofibers (Zhidkova et al., 1995; Van der Flier et al., 1995; Belkin et al., 1996; Belkin et al., 1997).

Mutations in the genes that encode the many components of the dystrophin glycoprotein complex cause the majority of muscular dystrophies. Mutations in the α7 gene also cause congenital myopathies (Hayashi et al., 1998). Thus, both the integrin- and dystrophin-mediated transmembrane linkage systems contribute to the functional integrity of skeletal muscle. Interestingly, there is an increase in the amount of α7 transcript and protein in DMD patients and mdx mice (the mouse model that has a mutation in its dystrophin gene) (Hodges et al., 1997). This led us to suggest that enhanced expression of the integrin may partially compensate for the absence of the dystrophin glycoprotein complex (Hodges, et al., 1997; Burkin and Kaufman, 1999). Utrophin, a protein homologous to dystrophin, is also increased in DMD patients and mdx mice (Law, et al., 1994; Pons et al., 1994). Utrophin associates with many of the same proteins as dystrophin, and further increasing utrophin may, in part, also compensate for the absence of dystrophin (Tinsley et al., 1996).

Although DMD patients (Monaco et al., 1987) and mdx mice (Bulfield et al., 1984; Sicinski, 1989) both lack dystrophin, the pathology that develops in the mdx mouse is much less severe than that observed in humans. The differences in the extent of pathology may be due to a number of factors including the enhanced expression and altered localization of utrophin (Law, et al., 1994; Pons et al., 1994) and the α7 integrin chain (Hodges et al., 1997) in mdx mice. In addition, differences in utilization of skeletal muscles by humans compared to mice in captivity may also contribute to the decreased level of pathology seen in mdx mice. In contrast, mdx/utr (−/−) mice lack both dystrophin and utrophin and have a phenotype that is similar to that seen in Duchenne patients. These double mutant mice develop severe progressive muscular dystrophy and die prematurely between 4-20 weeks of age (Grady et al., 1997b; Deconinck, et al., 1997b).

To explore the hypothesis that enhanced expression of the α7β1 integrin compensates for the absence of the dystrophin glycoprotein complex and reduces the development of severe muscle disease, transgenic mice were made that express the rat α7 chain. The mdx/utr (−/−) mice with enhanced expression of the α7BX2 chain isoform show greatly improved longevity and mobility compared to non-transgenic mdx/utr (−/−) mice. Transgenic mice maintained weight and had reduced spinal curvature (kyphosis) and joint contractures. Transgenic expression of the α7BX2 chain also reduced the degree of mononuclear cell infiltration and expression of fetal myosin heavy chain (fMyHC) in muscle fibers. Together these results show that enhanced expression of α7BX2β1D integrin significantly reduces the development of muscular dystrophy.

Muscle fibers attach to laminin in the basal lamina using the dystrophin glycoprotein complex and the α7β1 integrin. Defects in these linkage systems result in Duchenne muscular dystrophy, α2 laminin congenital muscular dystrophy, sarcoglycan related muscular dystrophy, and α7 integrin congenital muscular dystrophy. Therefore the molecular continuity between the extracellular matrix and cell cytoskeleton is essential for the structural and functional integrity of skeletal muscle. To test whether the α7β1 integrin can compensate for the absence of dystrophin, we have expressed the rat α7 chain in mdx/utr (−/−) mice that lack both dystrophin and utrophin. These mice develop a severe muscular dystrophy highly akin to that observed in Duchenne muscular dystrophy, and they also die prematurely. Using the muscle creatine kinase promoter, expression of the α7BX2 integrin chain was increased approximately 2.3-fold in mdx/utr (−/−) mice. Concomitant with the increase in the α7 chain, its heterodimeric partner, β1D, was also increased in the transgenic animals. The transgenic expression of the α7BX2 chain in the mdx/utr (−/−) mice extended their longevity by three-fold, reduced kyphosis and the development of muscle disease, and maintained mobility and the structure of the neuromuscular junction. Thus, bolstering α7β1 integrin-mediated association of muscle cells with the extracellular matrix alleviates many of the symptoms of disease observed in mdx/utr (−/−) mice and compensates for the absence of the dystrophin- and utrophin-mediated linkage systems.

There is a long felt need in the art for materials and methods for identifying compositions and/or conditions which increase the expression of α7 integrin and for definitive and accurate methods for the diagnosis of particular types of neuromuscular disorders, and for direct or indirect (e.g. drug) treatment. Enhanced expression of the α7β1 integrin provides a novel approach for and fulfills a long felt need for treatment of Duchenne muscular dystrophy and other muscle diseases that arise due to defects in the dystrophin glycoprotein complex.

### SUMMARY OF THE INVENTION

The present invention provides compositions and methods for ameliorating the physical condition and mobility of muscular dystrophy patients, for example, those suffering from Duchenne muscular dystrophy. There is also the beneficial result of longer life and better quality of life for patients treated according to the teachings and methods of the present invention. The present disclosure shows that expression over normal levels of the integrin polypeptide α7BX2 in muscle cells results in improved physical condition and mobility in the mouse model for Duchenne muscular dystrophy. Such overexpression also benefits individuals suffering from or susceptible to other forms of muscular dystrophy in which there is a deficiency in dystrophin and/or utrophin or α7 integrin. Similar improvements are achieved with the overexpression of the α7BX2 integrin polypeptide in human muscular dystrophy patients as well, either due to expression of an α7BX2 transgene specifically in muscle cells of human MD patients or due to increased expression of the naturally occurring gene due to stimulation of expression by the administration of a therapeutic composition with that effect. Human patients are similarly improved with respect to physical parameters and quality and length of life by the administration of compositions which improve the stability of the integrin protein. The expression of the α7BX2 coding sequence under the control of a muscle specific promoter in a human patient results in increased levels of the β1D polypeptide as well, with the result of increased function and quality of life. Any suitable vector for introducing the specifically regulated α7BX2 coding sequence can be used in the treatment of muscular dystrophy patients, with administration according to art-known methods. Intravenous or intramuscular administration or regional perfusion of a viral or plasmid vector comprising the muscle cell-specific expression construct is a desirable route of administration. Retroviral vectors, lentivirus vectors, adenovirus vectors and adeno-associated vectors are known and available to the art. Alternatively, the patient's myoblasts or stem cells can be harvested, transfected with a vector containing the muscle cell-specific expression construct, selected and expanded or ex vivo and then reintroduced into the patient by the intravenous route. Patients suffering from other forms of muscular dystrophy where α7 integrin protein levels are below normal similarly benefit from expression of an exogenous α7 coding sequence so that increased amounts of α7β1 protein are increased in muscle cells, with the result that the symptoms of muscular dystrophy are ameliorated.

As an alternative to the use of gene therapy to increase α7BX2 expression in the muscular dystrophy patient, one can administer a composition effective for enhancing the level of expression of the patient's own α7BX2 or other α7. The present invention provides methods for screening compositions or conditions for the ability to enhance α7BX2 expression: one of ordinary skill in the art can use quantitative (or semi-quantitative) reverse transcriptase-polymerase chain reaction (RT-PCR) assays or Northern hybridizations which allow determination of relative amounts of mRNA after administration of a test composition in comparison to a control lacking the test composition of interest. Alternatively, one can monitor expression of chimeric reporter molecules (including, but not limited to, green or other fluorescent protein, luciferase, β-galactosidase, β-lactamase, β-glucosidase, β-glucuronidase, chloramphenicol acetyl transferase), to evaluate drug-induced expression of the α7 integrin promoter linked to sequences encoding the reporter. The construction of particular chimeric reporter genes is provided below. As used herein, a reporter is a protein which can be quantified directly or via its enzymatic activity. Muscle cells or myogenic cells or myoblasts in culture transfected with the vector are treated with test compositions or conditions, and the amounts of α7BX2 or α7-regulated transcripts or reporter gene products are determined in response to small molecule test compositions in comparison to a control which has not treated with the small molecule test composition. As used herein, a small molecule is less than 2000 d. It can be a sugar, an oligosaccharide, a nucleotide or derivative, an oligonucleotide, a lipid, a peptide or any other small molecule, provided that it is not toxic to the cell in which expression is tested. Expression is enhanced in response to the test composition when the level of α7BX2 or α7-specific transcript is greater in the presence than in the absence of the test composition. Alternatively, the amount or relative amount of α7BX2 or α7 protein is determined after growth of the muscle or myogenic cells in the presence and absence of the test composition. The amount or relative amount can be determined using α7BX2 or α7-specific antibody using any of known immunological assays: radioactive immunoassay, western blotting, enzyme-linked immunoassays, sandwich immunoassays and the like. As an alternative to an immunological methods, the amount or relative amount of the protein can be determined by the use of muscle or myogenic cells transformed with a fusion protein coding sequence for an α7BX2 protein linked to a green fluorescent protein sequence, or enzymatic reporters such as luciferase, β-lactamase, β-galactosidase, or β-glucuronidase, among others, or an immunological tag portion or polyhistidine tag which can then allow specific immunological measurement of the target fusion protein. Such a fusion protein is expressed under the regulatory control of the native α7 promoter. Compositions identified by any of the assay methods noted above are used in the amelioration of muscular dystrophy symptoms by stimulating or increasing expression of the patient's own gene. The α7BX2-mdx/utr (−/−) mice can also be used for in vivo assays for compounds which ameliorate muscular dystrophy, by treating the mice with test compounds and observing an improvement in physical status or α7 expression.

In some human muscular dystrophies there are changes in the amounts of the α7A and α7B integrin protein isoforms. Detection of the α7A and α7B isoforms can be via immunological analysis, or it can be via specific hybridization using isoform specific primers for use in a reverse transcriptase polymerase reaction assay with the detection of the α7 integrin isoform amplification product of a specific size as described herein using the particular primers described herein, the α7A product is 451 bp whereas the amplification product produced from an α7B transcript is 338 bp in length.

In a method for assessing integrin expression in an individual, a sample of muscle tissue from the individual is provided and, if necessary, treated to render the components of the tissue available for antibody binding, the muscle tissue sample being characterized by levels of the α7A integrin protein; contacting the muscle tissue sample with an antibody which specifically binds to the α7A integrin protein, wherein said contacting under conditions appropriate for binding of the antibody to the α7A integrin protein; detecting the extent of binding of the antibody to the α7A integrin protein in the muscle tissue sample; and comparing the extent of binding of the antibody specific for the α7A integrin protein in the muscle tissue sample from the individual for whom diagnosis is sought to the extent of binding of the antibody specific for the α7A integrin protein in a muscle tissue sample from a normal individual, wherein a substantial reduction in the extent of binding of the antibody specific for the α7A integrin protein in the muscle tissue sample from the individual for whom diagnosis is sought as compared with the extent of binding in the muscle tissue sample of a normal individual is characteristic of an α7A integrin deficiency disorder. Desirably the muscle tissue samples are from skeletal muscle tissue. Histological specimens from an individual for whom diagnosis is sought and from a normal individual can also be used with antibody detection methods. Detection of the bound antibody can be via a detectable label such as a fluorescent compound, a chemiluminescent compound, radioactive label, enzyme label or other label known to the art, coupled with detection methods obvious in choice to one of ordinary skill in the art. A second antibody which recognizes the (first) integrin-specific antibody can be labeled and used to detect the bound first antibody. Advantageously, assays can be run in parallel for the assessment of the expression of 2/4 laminin in the individual for whom diagnosis is sought (and in a normal (control) sample.

The method can also be based on western blot analysis. In such a method the muscle tissue samples are solubilized, the components are separated by electrophoresis, for example, polyacrylamide gel electrophoresis or sodium dodecyl sulfate polyacrylamide gel electrophoresis, the separated components are transferred to a solid support to form an immunoblot, the immunoblot is contacted with antibody specific for the α7 integrin isoform under conditions appropriate for the binding of the antibody to the cognate integrin protein, the non-specifically bound material is removed, and the specific binding of the antibody to the α7 integrin isoform is detected, and the extent of the antibody binding to the immunoblot from the muscle tissue samples of the individual for whom diagnosis is sought is compared to the extent of antibody binding to an otherwise identical immunoblot prepared from a muscle tissue sample from a normal individual, wherein a substantial reduction in the extent of antibody binding to the α7 integrin protein isoforms in the immunoblot of the sample from the individual for whom diagnosis is sought as compared to the antibody binding in the immunoblot for the muscle tissue sample from a normal individual is indicative of an α7 integrin deficiency disorder. Desirably, the muscle tissue samples are from skeletal muscle.

Reverse transcriptase-polymerase chain reaction (RT-PCR) can also be carried out on muscle tissue samples from an individual for whom an assessment of α7A and α7B integrin expression is sought. RNA is extracted with precautions for preservation of messenger RNA in the samples. The primers noted below or other primers which result in the production of an amplification product characteristic in size of the α7A and α7B integrin messenger RNAs are used. Alternatively, Northern hybridizations can be carried out on RNA samples from muscle tissue specimens with probes characteristic of the α7 isoform transcript. The primers disclosed herein can be used in the general procedure as disclosed in Hayashi et al. (1998).

### BRIEF DESCRIPTION OF THE DRAWINGS

FIGS. 1A-1D illustrates the genotyping of transgenic α7BX2-mdx/utr (−/−) mice. FIG. 1A: The α7BX2 transgene (tg) was detected by PCR using primers that amplify between the MCK promoter and the α7 cDNA sequence. Lanes 2 and 3 are positive for the MCK-α7BX2 transgene. FIG. 1B: Southern analysis using a rat α7 specific probe of EcoRI and Kpnl digested genomic DNA. The 7.1 kb band corresponding to the rat transgene construct is detected in lanes 4, 5 and 6. A higher 14.2 kb transgene dimer was also detected. Samples in these lanes are from mdx/utr (−/−) mice. DNA in lanes 1, 2 and 3 are from non-transgenic mice. FIG. 1C: Determining the status of the utrophin gene by PCR. Only mutant utr alleles are detected in lanes 1 and 4 identifying utr (−/−) mice. One wildtype (wt) and one mutant allele are amplified in lane 2, identifying a utr (±) mouse. Lane 3 is wildtype at both utr loci. FIG. 1D: Determining the status of the dystrophin gene by PCR. The mdx primer set detects the point mutation in the dystrophin gene, whereas the wt primers detect only the wildtype allele. Mouse 2 is wildtype at the dystrophin locus, mouse 3 is heterozygous (mcW+) and mouse 4 is mdx. Lane 1 contains no DNA.

FIG. 2 demonstrates the expression of the rat α7 protein in mouse muscle. Immunofluorescence analysis of hindlimb cryosections using monoclonal antibodies against the rat α7 integrin chain, dystrophin, and utrophin. AChRs were stained with rhodamine-labeled α-bungarotoxin. The rat α7 protein is only detected in transgenic mice and localizes to the membrane of muscle fibers. The lack of dystrophin and utrophin in both transgenic and non-transgenic mdx/utr (−/−) mice confirms their genotypes.

FIG. 3 illustrates the immunofluorescence of β1 integrin isoforms in the hindlimb of 8 week wildtype, mdx, mdx/utr (−/−) and α7BX2-mdx/utr (−/−) mice. B1A integrin is elevated in muscle fibers of mdx/utr (−/−) mice compared to wildtype and mdx animals. In contrast, β1A levels are normal in α7BX2-mdx/utr (−/−) mice. Compared to wildtype, an increase in β1D is detected in both mdx and mdx/utr (−/−) muscle. α7BX2-mdx/utr (−/−) mice show an additional increase in β1D compared to both mdx and mdx/utr (−/−) mice.

FIGS. 4A-4C show the transgenic expression of α7BX2 increases the amount of β1D in hindlimb muscle. FIG. 4A: Western blot showing more α7B is detected in transgenic mice compared to non-transgenic mice whereas α7A is constant. FIG. 4B: The blots were re-probed with anti creatine kinase antibody. The CK levels were used to normalize the amounts of α7A and α7B proteins in each sample. The levels of α7A/CK in both transgenic and non-transgenic mice remained constant. In contrast, α7B/CK ratio is 2.3 fold higher in the α7BX2 transgenic mice compared to the non-transgenic animal. FIG. 4C: β1D integrin from 8 week hindlimb muscle. Less β1D is detected in mdx/utr (−/−) mice compared to α7BX2-mdx/utr (−/−) mice. An increase of approximately 1.5-fold more β1D was detected in the transgenic vs non-transgenic mice.

FIG. 5 provides Kaplan-Meier survival curves of 43 α7BX2-mdx/utr (−/−) and 84 mdx/utr (−/−) mice. Wilcoxon and Log rank tests show the α7BX2-mdx/utr (−/−) mice and mdx/utr (−/) populations have distinct survival curves (P<0.001). The α78X2-mdx/utr (−/−) mice survive 3-fold longer than non-transgenic mdx/utr (−/−) mice with a median life expectancy of 38 weeks. In contrast, non-transgenic mdx/utr (−/−) mice have a median life expectancy of just 12 weeks. 95% confidence intervals are indicated by shading.

FIG. 6 illustrates weight gain vs survival in representative mdx/utr (−/−) mice and α7BX2-mdx/utr (−/−) mice. The majority of non-transgenic mdx/utr (−/−) mice undergo a crisis at 5-10 weeks of age that results in a sudden loss of weight and premature death. Most transgenic mdx/utr (−/−) mice live longer and maintain weight. Eventually these also go through a crisis that results in weight loss.

FIG. 7 shows histology of hindlimbs from 10 week wildtype, mdx, mdx/utr(−/−) and αBX2-mdx/utr (−/−) mice. Hematoxylin and eosin staining reveal abundant central nuclei in mdx, mdx/utr (−/−) and α7BX2-mdx/utr (−/−) mice. Mononuclear cell infiltration and expression of fMyHC are extensive in the mdx/utr (−/−) mice, but are reduced in the α7BX2-mdx/utr (−/−) transgenic animals, indicating less degeneration and more stable regeneration in these mice.

FIG. 8 documents PCR detection of integrin α7A and α7B in normal control and SPMD patient samples. 35 cycles of amplification reveal minimal amounts of α7A in the SPMD patient samples.

FIG. 9A illustrates homologous recombination of the chimeric luciferase gene and neomycin resistance gene. Stable transfectants that arise by homologous recombination are selected as described below. FIG. 9B illustrates a random insertion of the luciferase and neomycin resistance sequences that will likely also contain the TK gene. Such TK-containing recombinants are selected against using the nucleoside analog gangcyclovir.

FIG. 10 is a schematic illustration of a α7-regulated reporter gene vector.

### DETAILED DESCRIPTION OF THE INVENTION

Mutations in the α7 integrin gene resulting in the absence or reduction of the α7 integrin protein have been shown to be responsible for the myopathy and delayed motor milestones of 3 Japanese patients with previously undefined muscular dystrophies (Hayashi et al., 1998). In addition, expression of the α7β1 integrin protein has been shown to be up-regulated in Duchenne muscular dystrophies (DMD) and down-regulated in laminin-2/4 (α2β1y1)-deficient patients. Because of the role of the α7β1 integrin in muscle development, structure and function, we have further examined of its involvement in human muscle disease. Laminin-2/4 is also known as merosin. The structural gene encoding the α7 integrin has been mapped by fluorescence in situ hybridization (FISH) and radiation hybrid mapping to human chromosome 12q13.

Because of the diminished physical capacities and the early death of muscular dystrophy patients, especially Duchenne muscular dystrophy patients, there is a strong need for effective treatment of these individuals. Successful treatment has humanitarian advantages, as well as economic benefits to society and to families of affected individuals. It has been discovered that expression of the integrin polypeptide α7BX2 in muscle cells at greater than normal levels results in improved function and lifespan in the animal model for Duchenne muscular dystrophy (the mdx/utr (−/−) mouse). Treatment of human patients with genetic material containing a similarly regulated coding sequence for the integrin polypeptide α7BX2 results in improved physical condition and mobility as well as increased lifespan.

To confirm that the α7β1 integrin linkage system can alleviate severe muscle disease, transgenic mice were produced that express the rat α7 chain in a genetic background which resulted in the absence of dystrophin and utrophin. DNA encoding the rat α7 integrin α7BX2 isoform, under the transcriptional control of the mouse muscle creatine kinase (MCK) promoter, was cloned and shown to have biological activity in vitro (Burkin et al., 1998). The 3.3 kb MCK promoter limits transcription to differentiated skeletal and cardiac muscle, confining the effects of overexpression to these tissues (Donoviel et al., 1996). The 7.1 kb construct, MCK-α7BX2, was used to express the rat integrin in mdx/utr (−/−) mice. Due to the mortality of the double knockout mice, the rat transgene was initially introduced into a heterozygous [mdx/utr (±)] background and these animals were then bred to produce double knockout transgenic offspring. The ratio of offspring followed expected Mendelian genetics indicating the transgenic expression of the rat α7 integrin did not have an obvious effect on embryonic development.

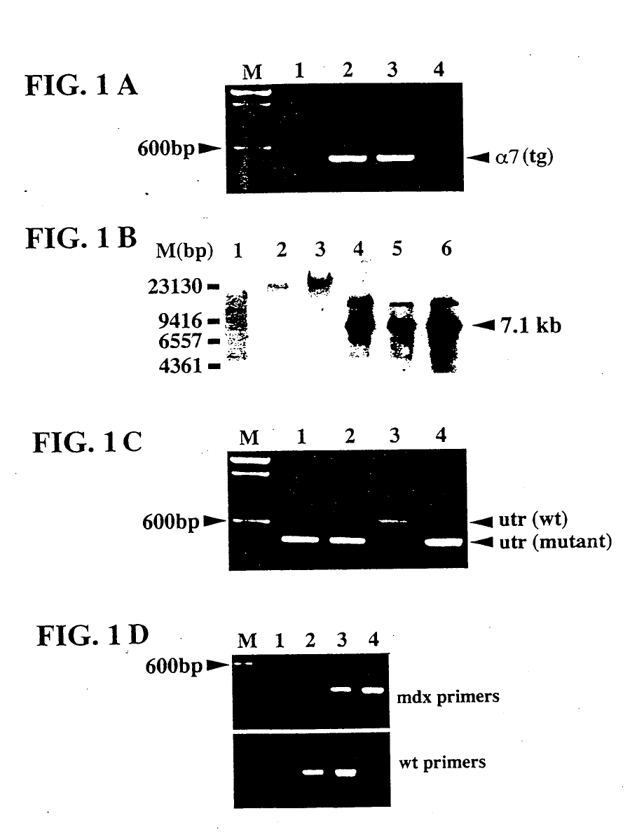

The presence of the rat α7 transgene was detected by both PCR and Southern analyses. Using MCKI and AATII primers, a 455 bp product was amplified only in transgenic mice (FIG. 1A). Southern analysis produced a strong 7.1 kb band only in transgenic mice. This is the expected size of the EcoRI and Kpnl digested MCK-α7BX2 construct (FIG. 1B). A weak 14.2 kb band was also detected by Southern analysis, suggesting a portion of the constructs had lost one of these restriction sites.

The status of the utrophin gene was analyzed by PCR using the primers 553, 554 and 22803 previously described (Grady et al., 1997a). A 640 bp product is amplified when the wildtype utrophin allele is present, whereas a 450 bp product is amplified when the utrophin mutant allele is present (FIG. 1C).

The status of the dystrophin gene was determined by the amplification resistant mutation detection system (Amalfitano and Chamberlain, 1996). Using the mdx-specific primer set, a 275 bp mutant allele is detected, while in separate reactions the wild type specific primer set detected a 275 bp wildtype allele. FIG. 1D shows three different genotypes at the dystrophin locus. Mouse 2 is wildtype at the dystrophin locus, mouse 3 is heterozygous (mdx −/+) while mouse 4 is mdx.

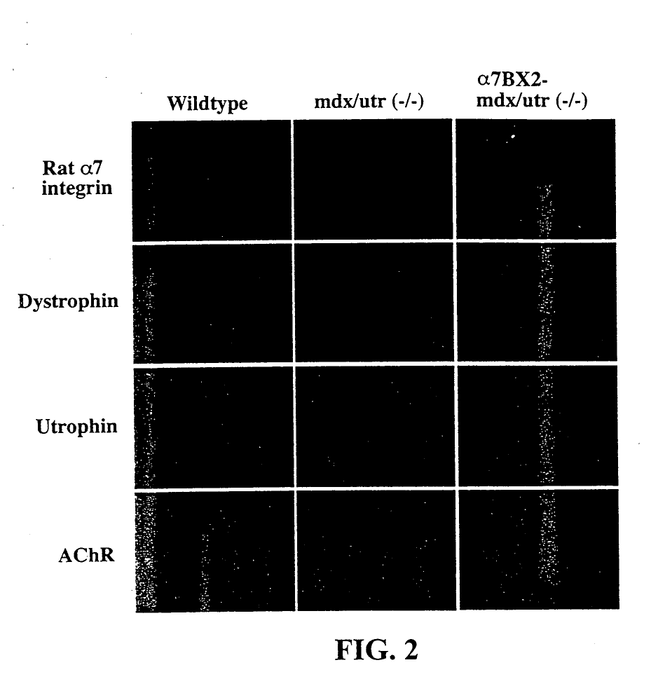

Protein expression from the rat α7 chain transgene was determined by immunofluorescence analysis of cryosections using the rat-specific α7 monoclonal antibody 026 (FIG. 2). The rat α7 chain was only detected by immunofluorescence in the muscle of transgenic mice (FIG. 2). Immunofluorescence also showed the absence of dystrophin in muscle fibers and the absence of utrophin at neuromuscular junctions in both transgenic and non-transgenic mdx/utr (−/−) mice (FIG. 2).

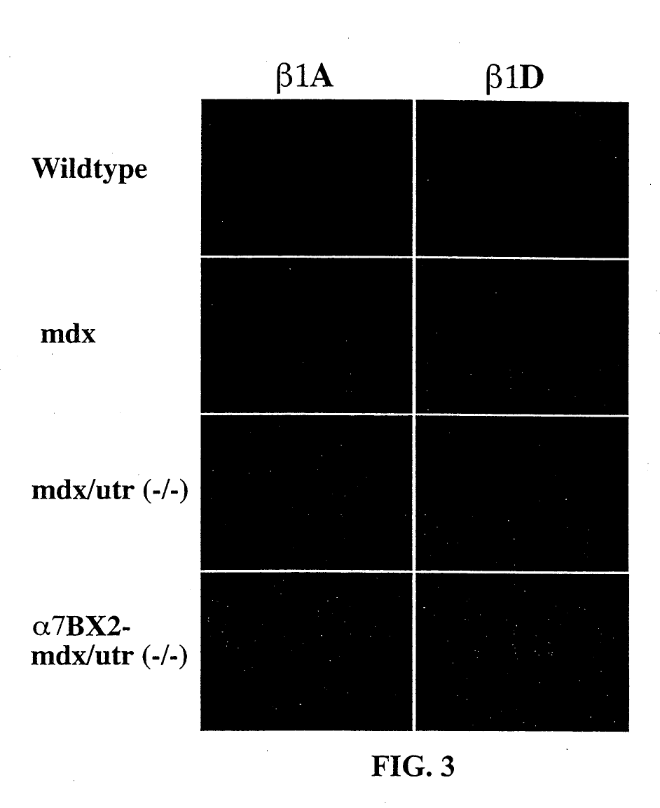

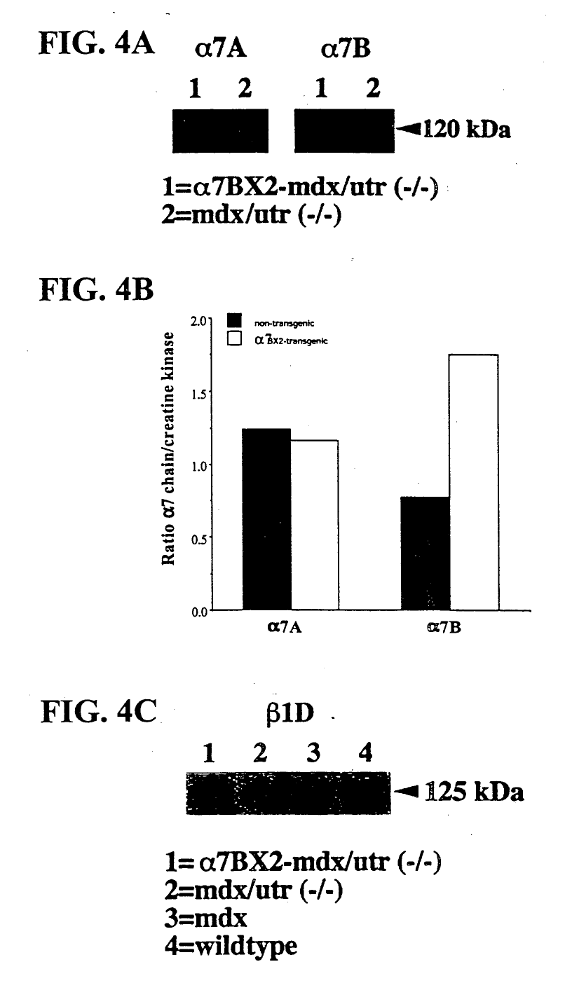

The alternative spliced form of the β1 integrin chain, β1D, is expressed in differentiated skeletal and cardiac muscle (Zhidkova, et al., 1995; Van der Flier, et al., 1995; Belkin, et al., 1996). Compared to the β1A, β1D may form stronger linkages between the cell cytoskeleton and extracellular matrix (Belkin et al., 1997). Immunofluorescence analysis showed β1A levels were elevated in fibers of mdx/utr (−/−) mice compared to wildtype and mdx animals. This is indicative of muscle that is not fully differentiated. In contrast α7BX2-mdx/utr (−/−) mice had normal levels of β1A integrin. Immunofluorescence and western blot analysis showed that mdx and mdx/utr (−/−) mice have more cell surface β1D chain than wildtype mice. This increase in β1D coincided with an increase in endogenous α7 chain in non-transgenic mdx and mdx/utr (−/−) mice as well total α7 in α7BX2-mdx/utr (−/−) mice. The α7BX2-mdx/utr (−/−) mice also had an additional 1.5-fold more β1D compared to mdx/utr (−/−) mice (FIGS. 3 and 4C). Thus an increase in the α7BX2β1D integrin is promoted by increased expression of the α7 transgene expressed specifically in muscle cells.

As previously reported, mdx mice express approximately two-fold more α7 integrin mRNA than wildtype controls (Hodges, et al., 1997). No further increase in α7 protein was detected in the mdx/utr (−/−) animals. The amount of α7BX2 protein in the α7BX2-mdx/utr (−/−) mouse hindlimb detected by western blots was approximately 2.3-fold greater than the endogenous α7BX2 chain in mdx/utr (−/−) mice (FIGS. 4A and 4B). As expected, the levels of α7AX2 were equivalent in the transgenic and non-transgenic mice.

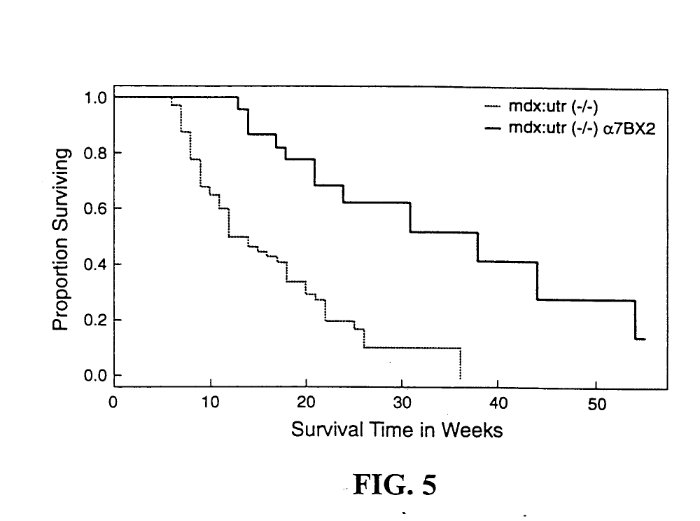

α7BX2-mdx/utr (−/−) mice exhibit increased longevity and mobility as compared to the mdx/utr (−/−) mice. Longevity was significantly extended in the α7BX2-mdx/utr (−/−) transgenic mice (FIG. 5). Kaplan-Meier survival analysis (Kaplan and Meier, 1958) of 84 non-transgenic and 43 transgenic mdx/utr (−/−) mice demonstrated that the observed differences in survival of these populations were statistically significant (p<.001). Log-rank (Peto et al., 1977) and Wilcoxon rank-sum tests (Conover, 1980) showed that the difference in survival emerged soon after birth and was maintained throughout the observed lifetime of the animals. The mdx/utr (−/−) mice used in these experiments developed severe muscular dystrophy and 50% died before 12 weeks of age. The median age at death of the transgenic mdx/utr (−/−) mice was 38 weeks, a three-fold increase over that observed in non-transgenic mdx/utr (−/−) littermates. These findings were similar in male and female mice. The oldest α7BX2-mdx/utr (−/−) mouse was sacrificed at 64 weeks of age.

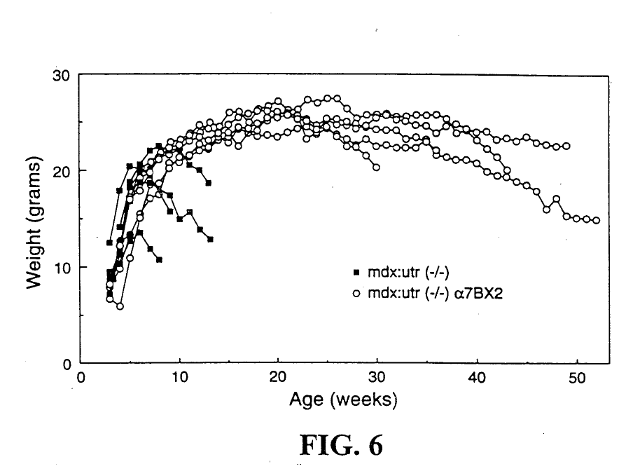

Compared to mdx mice that exhibit minimal pathology, mdx/utr (−/−) mice do not maintain weight. Instead these mice undergo a crisis period that results in weight loss and premature death at 8-20 weeks of age (Grady, et al., 1997b; Deconinck, et al., 1997b). In contrast, α7BX2-mdx/utr (−/−) transgenic mice did not show sudden weight loss. Animal weight stabilized between 20-25 grams (FIG. 6). No significant differences were found in the weights of mdx mice compared to α7BX2-mdx mice between 3 to 30 weeks of age. Thus, extra α7BX2 chain itself does not promote weight gain.

By 8 weeks of age mdx/utr (−/−) mice exhibited limited mobility and a waddling gait. In contrast, α7BX2-mdx/utr (−/−) littermates had highly improved mobility, comparable to mdx mice. The transgenic mice are dramatically improved in parameters including kyphosis (severe curvature of the spine), constriction of the rib cage, gait, joint contractures and mobility, as compared with the mdx/utr (−/−) mice lacking the transgene.

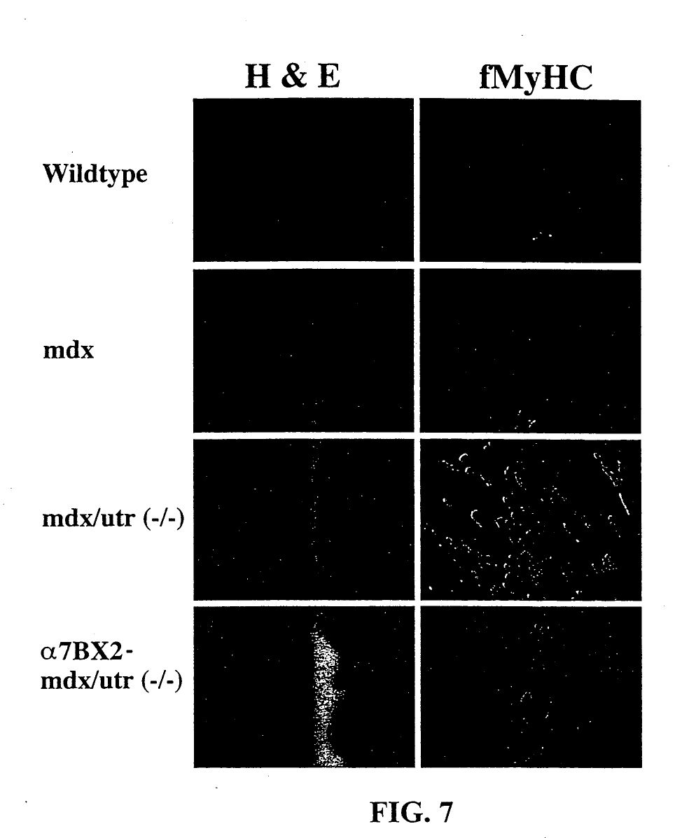

Enhanced expression of the α7BX2 chain stabilizes regeneration in mdx/utr (−/−) mice. Nuclei are normally localized along the periphery of myofibers, whereas in regenerating muscle nuclei are centrally located (DiMario, et al., 1991). Regeneration is also accompanied by a transient reversion to expression of fetal isoforms of myosin heavy chain (fMyHC) (Matsuda, et al., 1983; Sand, et al., 1987). Hindlimb sections from 5, 8 and 10 week old wildtype, mdx, mdx/utr (−/−) and α7BX2-mdx/utr (−/−) mice were stained with hematoxylin and eosin to determine the extent of mononuclear infiltration and centrally located nuclei (FIG. 7 and Table 1). Immunofluorescence of fMyHC was also determined. Degeneration and regeneration that are characteristic of muscle disease occur earlier in mdx/utr (−/−) animals compared to mdx mice (FIG. 7 and Table 1). These results are consistent with the earlier onset of necrosis and cell infiltration previously reported in these animals (Grady et al., 1997b; Deconinck et al., 1997b). The occurrence of central nuclei in α7BX2-mdx/utr (−/−) mice was similar to that in mdx/utr (−/−) mice indicating that enhanced expression of the integrin does not prevent early degeneration and regeneration. Likewise, fMyHC expression was most extensive at 5 weeks in the mdx/utr (−/−) and α7BX2-mdx/utr (−/−) mice. In contrast, mdx mice exhibited very little fMyHC at 5 weeks. At 8 weeks fMyHC was elevated in mdx mice and at 10 weeks it was reduced, indicating that a cycle of degeneration and regeneration was followed by stabilization. The shift from the 1A to β1D chain supports this conclusion. At all ages examined, the extent of fMyHC expression in the α7BX2-mdx/utr (−/−) animals was intermediate between that found in the mdx and mdx/utr (−/−) animals. In the 8 and 10 week old transgenic mdx/utr (−/−) mice, fMyHC expression approached that in mdx mice (FIG. 7). This decreased expression of fMyHC in α7BX2-mdx/utr (−/−) mice paralleled the greater integrity of tissue seen in the 8 and 10 week transgenic animals compared to the mdx/utr (−/−) mice. The extensive mononuclear cell infiltration observed in the mdx/utr (−/−) mice was also partially reduced in the α7BX2-mdx/utr (−/−) animals (FIG. 7). Thus, enhanced expression of the α7β integrin does not alter the initial degenerative cycle, but once regeneration has taken place, the additional integrin appears to stabilize muscle integrity reducing muscle pathology.

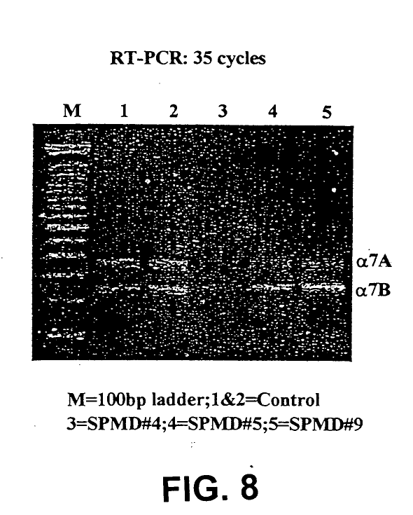

Kyphosis and joint contractures are alleviated in α7BX2-mdx/utr (−/−) mice as compared with the mdx/utr (−/−) mice. Severe curvature of the spine (kyphosis) in DMD patients and mdx/utr (−/−) mice is due to a failure of the muscles that would normally support the spinal column (Oda et al., 1993). X-ray images showed that both kyphosis and rib cage compression were markedly reduced in α7BX2-mdx/utr (−/−) mice compared to mdx/utr (−/−) littermates (FIG. 8). This was confirmed by whole body magnetic resonance imaging (MRI) which visualized not only the tissues surrounding the spinal column, but bundles of muscle fibers, the heart, lung and other soft tissues. The reduction in kyphosis promoted by the enhanced expression of integrin in the α7BX2-mdx/utr (−/−) animals likely is a major factor in their survival. Kyphosis results in the diaphragm being pushed forward, compromising lung capacity and diaphragm function, and thereby contributing to cardiopulmonary failure. A partial reduction of kyphosis has dramatic effects on survival.

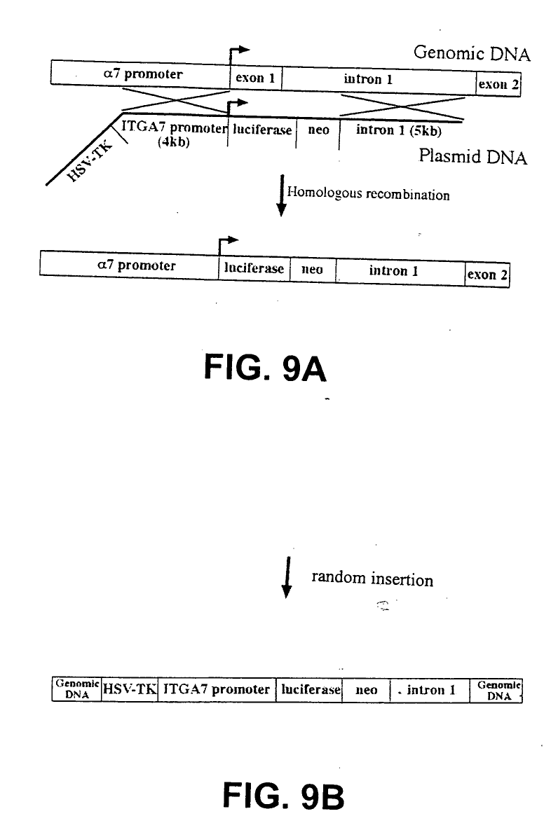

A hallmark of diseased musculature is the failure to extend limb muscles, resulting in joint contractures and impaired mobility. Hindlimb joint contractures are conspicuous in mdx/utr (−/−) mice but are markedly reduced in the α7BX2-mdx/utr (−/−) mice (FIG. 9). The reduction in hindlimb joint contractures allows the mice to have greatly improved mobility.

Structural changes from the normal patterns in the neuromuscular junctions of α7BX2-mdx/utr (−/−) mice are reduced due to the expression of the integrin chain. The neuromuscular junctions (NMJs) in utr (−/−) mice exhibit a significant reduction the numbers of synaptic folds, and density of AchRs (Grady et al., 1997a; Deconinck et al., 1997a). This is exacerbated in mdx/utr (−/−) mice that show even greater reductions in post-synaptic folding and AChR density (Grady et al., 1997b; Deconinck et al., 1997b). The post-synaptic plate of the NMJ in the mdx/utr (−/−) mice appears en face as discrete boutons rather than as a continuous folded NMJ structure (Grady et al., 1997b; Rafael et al., 2000). When the wildtype, mdx/utr (−/−) and α7BX2-mdx/utr (−/−) mice were examined with respect to localization of acetylcholine receptors (AChRs) detected with rhodamine-labeled α-bungarotoxin in the postsynaptic membrane, it was found that in wildtype mice, the postsynaptic membrane is continuous and uninterrupted. In contrast, mdx/utr (−/−) mice have discontinuous distributions of AChRs organized into discrete “boutons”. The organization of the postsynaptic membrane in α7BX2-mdx/utr (−/−) transgenic mice has a more continuous (normal) en face pattern.

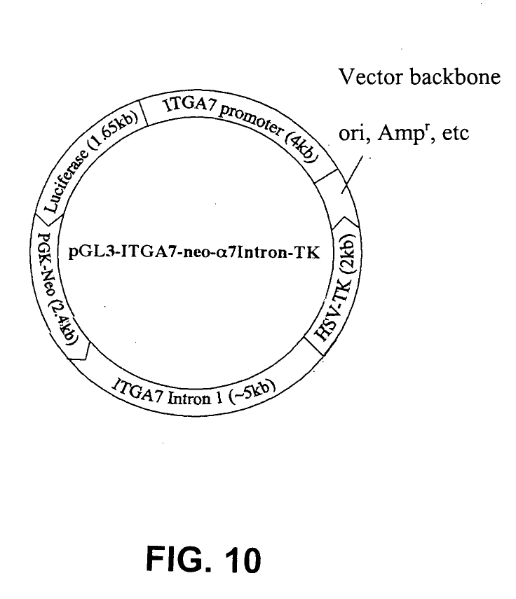

Because the α7β1 integrin is normally found at NMJs (Martin et al., 1996) and participates in the clustering of AChRs in C2C12 cells (Burkin et al., 1998, 2000), we compared the structure of NMJs from 8 week old wildtype, mdx/utr (−/−) and α7BX2-mdx/utr (−/−) mice (FIG. 10). Longitudinal sections from the hindlimb muscle were stained with rhodamine-labeled α-bungarotoxin and images of en face sections of the postsynaptic membrane were analyzed. Immunofluorescence staining of the NMJs of mdx/utr (−/−) mice appeared less intense than those of wildtype mice and showed extensive discrete boutons. In contrast, most NMJs from α7BX2-mdx/utr (−/−) mice appeared more continuous. Thus, enhanced levels of the α7β1 integrin help maintain the normal structure of the NMJ.

Our results demonstrate, for the first time, that enhanced expression of the α7β1 integrin can alleviate the development of muscular dystrophy and significantly extend longevity. Mice lacking both dystrophin and utrophin were used in this study because in the absence of both proteins, direct substitution of dystrophin with utrophin is precluded. This results in the development of severe muscular dystrophy and premature death, symptoms that closely resemble those seen in Duchenne muscular dystrophy (Grady, et al., 1997b; Deconinck, et al., 1997b), an important muscular dystrophy in humans.

The α7BX2-mdx/utr (−/−) mice reported here have approximately 2.3-fold more α7BX2 chain than their non-transgenic littermates. The βID chain, partner to α7, is also increased in the α7BX2 transgenic mice. The increased levels of α7β1 integrin led to a three-fold extension in median survival time, markedly improved mobility, and reduced kyphosis and joint contractures in the transgenic mdx/utr (−/−) mice. Kaplan-Meier survival analysis of the transgenic and non-transgenic mdx/utr (−/−) mice shows that the extension of longevity due to expression of the transgene is statistically significant and is evident early and throughout the life of the animals.

The survival times of the mdx/utr (−/−) mice in these experiments differ slightly from those previously reported. The original reported longevity of the mdx/utr (−/−) used to produce the animals in our experiments was 4-14 weeks (Grady, et al., 1997b). More recently, a life span of 4-20 weeks has been reported (Grady et al., 1999) and occasional longer living mice have been noted by others. We too have noted some “outliers” in that 6 of 84 mdx/utr (−/−) mice survived beyond 22 weeks, with the oldest mouse dying at 36 weeks of age. The transgenic and non-transgenic mice with extended life spans were re-evaluated for expression of dystrophin and utrophin by PCR and immunofluorescence and were again found deficient in both. Nevertheless, α7BX2-mdx/utr (−/−) mice are clearly distinct in longevity, mobility and histology from non-transgenic littermates. The median lifespan of the α7BX2-mdx/utr (−/−) mice was 38 weeks whereas the median life span for those not receiving the transgene was 12 weeks of age.

Electron microscopy has been used to compare the NMJs and myotendinous junctions of mdx/utr (−/−) and α7BX2-mdx/utr (−/−) mice. The normal folded morphology of the post synaptic membrane of then NMJ that is severely compromised in the mdx/utr (−/−) mice is largely maintained where there is increased expression of the α7BX2 integrin. Similarly, the normal folding of the myotendinous junction that is absent in the severely dystrophic mice is also maintained when the levels of the a7β1 integrin are increased. Thus, morphology of those structures that are involved in initiating muscle contraction and generating force and movement are preserved by enhanced expression of the integrins. Without wishing to be bound by any particular theory, the present inventor believes that the maintenance of the structure and function of both the myotendinous junction and neuromuscular junctions contributes to the increase in the lifespan of the transgenic mice.

Enhanced (increased) expression of integrin prevents development of cardiomyopathy. The elevation of atrial natiurectic factor (ANF) seen in dystrophic mdx/utr (−/−) mice (and in dystrophic humans) is largely alleviated in animals expressing elevated levels of the α7β1 integrin. Likewise, uptake of Evans blue, an indicator of membrane damage, and histologic determination of lesions in the heart all indicate that cardiomyopathy is largely reduced in the α7BX2-mdx/utr (−/−) mice as compared with the double knockout animals. Thus, enhanced expression of the integrin significantly prevents the development of pathology in both skeletal and cardiac muscle and it alleviates then symptoms in humans or animals suffering from the symptoms of dystrophy.

Although the mechanism by which enhanced expression of the α7 integrin protein alleviates the development of the dystrophin-deficient phenotype is not currently understood, multiple effects that result from additional β and β integrin chains are possible. An added advantage of the α7BX2 integrin expression is that it is a protein produced in the muscular dystrophy patients, and therefore, there is no potential for an immune reaction to it as there would be in the recombinant expression of a protein which is not already expressed in those patients.

Suitable vectors for directing the expression of the α7BX2 integrin expression include retrovirus vectors, adenovirus vectors and adeno-associated virus vectors. Vectors and methods are described in references including, but not limited to, Campeau et al. (2001); Stedman, H. (2001); Yoon and Lee (2000); Wang et al. (2000), Ragot et al. (1993), Muzyczka, N. (1992), Greelish et al. (1999), Xiao et al. (2000), Cordier et al. (2000), Ascadi et al. (1996), Gilbert et al. (1999), Ebihara et al. (2000), Fujii et al. (2000), Poirier et al. (2000).

Expression of the β1D chain is, in nature, restricted to differentiated skeletal and cardiac muscle (Zhidkova, et al., 1995; Van der Flier, et al., 1995; Welkin, et al., 1996; Welkin et al., 1997). In contrast, the β1A chain is present in a wide variety of cell types including myogenic precursor cells. The β1D cytoplasmic domain acts to arrest the progression of myoblast proliferation, alter subcellular localization and affinity of α7β1 for its ligand, and alter the association of the α7βl with the cell cytoskeleton (Welkin et al., 1997).

Increased β1D expression in α7BX2 transgenic mice appears to increase the interaction between the extracellular matrix, sarcolemma and the cell cytoskeleton, stabilizing muscle integrity. Moreover, β1A, characteristic of non-muscle cells and undifferentiated muscle, is increased in mdx/utr (−/−) and decreased in the transgenic mdx/utr (−/−) animals. The shift from β1A and increased β1D reflects less mononuclear cell infiltrates and increased stability of muscle fibers in the rescued mice.

The α7BX2 integrin chain is normally concentrated at neuromuscular and myotendinous junctions (Martin et al., 1996), as well as at intrafascicular junctions. In patients with Duchenne muscular dystrophy and in mdx and mdx/utr (−/−) mice, endogenous expression of the α7 integrin protein is increased and the α7BX2 isoform is also found extrajunctionally (Hodges et al., 1997). This increase in expression and re-distribution of α7β1 integrin in dystrophic mice is also seen with utrophin that is normally confined to neuromuscular junctions (Matsumura et al., 1992). Immunolocalization of integrin encoded by the rat α7 transgene, detected with anti-rat α7 antibodies, shows that the rat α7 protein is also distributed more globally in the α7BX2-mdx/utr (−/−) animals. Enhanced expression of the integrin therefore contributes to the mechanical integration and stability between muscle fibers and at their junctional sites. Other possible mechanisms may also underlie how the α7β1 integrin rescues mdx/utr (−/−) mice.

Whereas the MCK promoter drives transcription in skeletal and cardiac muscle (Donoviel et al., 1996), enhanced expression of the αβ1 integrin in the heart also contributes to the rescue of these animals. However, expression of utrophin in skeletal muscle, but not cardiac muscle, of mdx/utr (−/−) mice increased survival and reduced pathology (Rafael et al., 1998). These observations suggest that the loss of skeletal muscle integrity is the major factor in the development of muscle pathology in mdx/utr (−/−) mice.

The role of the α7β1 integrin in the formation of the postsynaptic membrane (Burkin, et al., 1998; 2000) suggests that increased integrin expression enhances the development and stability of the NMJ. Dystrophin and utrophin are also concentrated at the postsynaptic membrane and mdx/utr (−/−) and mdx/utr (−/−) mice show progressive alterations of the ultrastructure of these sites (Grady et al., 1997b; Deconinck et al., 1997b). Whereas wildtype and utr (−/−) mice have NMJ endplates that are highly folded and continuous, mdx and mdx/utr (−/−) mice show discontinuous NMJs that are described as discrete “boutons” (Grady et al., 1997a; 1997b; Rafael et al., 2000). Whereas both mdx and utr (−/−) mice show a reduction in the number of synaptic folds when compared to wildtype mice, mdx/utr (−/−) mice show even fewer synaptic folds (Grady et al., 1997b; Deconinck et al., 1997b). Transgenic expression of the α7BX2 chain appears to maintain the normal en face structure of the postsynaptic membrane in mdx/utr (−/−) mice.

In the absence of dystrophin, there is an increase in total muscle calcium (Bertorini et al., 1982) and an elevation of intracellular calcium ([Ca²⁺]i) in isolated dystrophic myofibers (Turner et al., 1988). These increases have been attributed to leaky calcium channels in dystrophic muscle compared to normal muscle. The [Ca²⁺]i increase may activate Ca²⁺-dependent proteolysis and increase muscle degeneration (Denetclaw et al., 1994). [Ca²⁺]i levels are also regulated by signaling through the α7β1 integrin (Kwon et al., 2000), suggesting that this integrin may contribute to the maintenance of calcium levels in myofibers. If so, the transgenic expression of the α7BX2 chain may regulate the activity of calcium channels, stabilizing [Ca²⁺]i levels in mdx/utr (−/−) myofibers and reducing Ca²⁺-dependent proteolysis and muscle degeneration.

Enhanced expression of the α7 integrin may contribute to additional changes in the expression of other proteins, both within the cell and in the extracellular matrix. For example, matrix stability or modeling may potentiate both mechanical and signal transduction capacities of muscle (Colognato et al., 1999). This dual role for the integrin is consistent with analyses of α7 (−/−) mice. The myotendinous junctions of fast fibers are compromised in α7 deficient mice (Mayer, et al., 1997). These myofibers also exhibit a partial shift from β1D to βIA integrin and activation of the c-Raf-l/mitogen-activated protein kinase-2 signaling pathway. These changes cause a reduction of integrin-dependent association of fibers and the basal lamina, contributing to the dystrophy that develops in these mice (Saher and Hilda, 1999). As shown herein, increased α7 chain leads to increased β1D integrin.

A broad phenotype is seen in children with congenital muscular dystrophies that arise from mutations in the α7 gene (Hayashi et al., 1998). These patients exhibit congenital myopathy, delayed motor milestones, and severe impairment of mobility. These phenotypes are consistent with a role for α7β1 integrin in the formation and stability of the postsynaptic membrane, myotendinous junctions, and overall stability of muscle integrity.

Since enhanced expression of the α7β1 (or other α7) integrin can alleviate many of the symptoms of severe muscular dystrophy in mdx/utr (−/−) mice, it appears that the integrin-mediated and dystrophin-mediated linkage systems between myofibers and the extracellular matrix are in many ways functionally complementary mechanisms. As such, the enhanced expression of the α7β1 or other α7 integrin is a novel approach to alleviate Duchenne muscular dystrophy and treat α7 integrin-deficient congenital muscular dystrophies. Moreover, increasing integrin levels proves effective in reducing the development of other muscular dystrophies and cardiomyopathies that arise from compromised expression of other components of the dystrophin glycoprotein complex, but especially those muscular dystrophies in which there is a lower than normal level of α7 integrin protein.

As an alternative to the use of gene therapy to increase α7BX2 or other α7 expression in the muscular dystrophy patient, one can administer a composition effective for enhancing the level of expression of the patient's own α7BX2 or other α7 sequence. The present invention provides methods for screening for enhanced α7 expression: one of ordinary skill in the art can use quantitative (semi-quantitative) reverse transcriptase-polymerase chain reaction (RT-PCR) assays or Northern hybridizations which allow determination of relative amounts of mRNA. Muscle cells or myogenic cells (either normal or derived from a muscular dystrophy patient or from an animal model for same) in culture are treated with test compositions and the amounts of α7BX2 or α7-specific transcripts are determined in response to test compositions in comparison to a control which has not treated with the test composition. Expression is enhanced in response to the test composition when the level of α7BX2 or α7-specific transcript is greater in the presence than in the absence of the test composition. Alternatively, the amount or relative amount of α7BX2 or other α7 protein is determined after growth of the muscle or myogenic cells in the presence and absence of the test composition. The amount or relative amount can be determined using α7BX2 or α7-specific antibody using any of known immunological assays: radioactive immunoassay, western blotting, enzyme-linked immunoassays, sandwich immunoassays and the like. As an alternative to immunological methods, the amount or relative amount of the protein can be determined by the use of muscle or myogenic cells transformed with a fusion protein coding sequence for an α7BX2 or other α7 protein linked to a green or other fluorescent protein sequence, other reporters (such as luciferase, β-galactoside, β-lactamase, β-glucuronidase, among others) or an immunological tag portion which can then allow specific immunological measurement of the target fusion protein. Such a fusion protein is expressed under the regulatory control of the native α7 promoter. Compositions identified by any of the assay methods noted above are used in the amelioration of muscular dystrophy symptoms by stimulating or increasing expression of the patient's own gene. Similarly, screening can be accomplished in which increased levels of the polypeptide are detected in response to treatment of the cells with a composition which increases the stability of the α7BX2 or other α7 protein in the cells. Compositions identified by the screening methods described herein are useful in vivo for the increased expression and/or stability of the α7BX2 or other α7 protein in muscle cells and for the amelioration of muscular dystrophy symptoms in patients due to a net increase in the α7BX2 or other α7 protein. Methods for high throughput screening for expression levels or for the amount of a fluorescence-tagged or enzyme-tagged protein are well known in the art, and can be readily adapted to the present measurement of α7BX2 or other α7 protein without the expense of undue experimentation.

Altered expression of the α7β1 integrin is evident at a relatively high frequency in patients with muscular dystrophies of undefined origin. To determine the extent of involvement of the α7β1 integrin in skeletal muscle diseases, 303 human biopsy samples were screened for expression of both the α7A and α7B isoforms. Of these, 36 patients were totally deficient in both isoforms, whereas the others had anomalous expression of only one isoform of the α7 chain. This indicates that complex regulation of integrin production, or selective stability, underlies certain muscle diseases. The high frequency of involvement of the α7β1 integrin in congenital muscle diseases supports the need for rapid screening and analyses of patients.

The β1D integrin isoform is the heterodimeric partner of α7 integrin in skeletal and cardiac muscle. Because of the altered expression of the α7 integrin in certain patients, one can examine a patient of interest to determine if expression of the β1 integrin protein is also affected.

Monoclonal or polyclonal antibodies, preferably monoclonal, specifically reacting with a particular integrin subunit polypeptide or encoded by a particular coding sequence, especially an α7β1 or other α7 integrin, have been made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience, New York, N.Y.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

All references cited in the present application are incorporated by reference herein to the extent that there is no inconsistency with the present disclosure.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1

### MCK-α7BX2 Integrin Construct

The CDNA encoding the rat α7BX2 integrin isoform was cloned into the pBK-RSV vector (Stratagene, La Jolla, Calif.) downstream of the 3.3 kb mouse muscle creatine kinase promoter (MCK, described in Jaynes et al., 1986) and the mouse α7 integrin cell surface localization signal sequence using the restriction sites Aatil and Kpnl. The MCK promoter was kindly provided by Dr. Stephen Hauschka, (University of Washington). The construct was verified by DNA sequencing. Previous studies have shown that the MCK promoter is only active in heart and skeletal muscle (Jaynes et al., 1986; Johnson et al., 1989; Shield et al., 1996). The expression and functionality of the MCK-α7BX2 integrin construct was verified by transfecting C2C12 myoblasts (Burkin et al., 1998; Burkin et al., 2000). The sequence of the integrin α7 subunit is given in Song et al. (1992). See also Burkin and Kaufman (1998) for a discussion of the MCK-regulated construct.

### Example 2

### Production of Transgenic mdx/utr(−/−) mice

The MCK-α7BX2 construct-containing DNA fragment was gel purified. Fl female mice from a C57BL6 X SJ6 strain cross were superovulated, mated to Fl male mice and fertilized oocytes were collected. The MCK-α7BX2 construct was microinjected into male pronuclei and injected oocytes were placed into pseudopregnant mice at the University of Illinois Transgenic Animal Facility. Resulting pups were weaned at 3 weeks of age. Genomic DNA was isolated from 0.5 cm tail clips using a DNA isolation kit (Promega, Madison, Wis.). Primers (MCK1: 5′-caagctgcacgcctgggtcc-3′, SEQ ID NO:1; and AATII: 5′-ggcacccatgacgtccagattgaag-3′, SEQ ID NO:2) used to amplify between the MCK promoter and the α7 integrin cDNA resulted in a 455 bp amplimer only in transgenic mice. Transgenic male Fl mice were bred with mdx/utr (±) female mice, provided by Dr. Joshua Sanes (Washington University, St. Louis, Mo.).

All male offspring were mdx due to the location of the dystrophin gene on the mouse X-chromosome. The mdx mutation was also screened by the amplification resistant mutation system described by Amalfitano and Chamberlain (1996). A new forward primer (Int22-306F, 5 ′-catagttattaatgcatagatattcag-3′, SEQ ID NO:3), upstream of the mdx mutation site was used to yield a larger, 275 bp band. The status of the utrophin gene was analyzed by PCR using the primers 553, 554 and 22803 previously described by Grady et al., (1997a). Transgenic mdx/utr (±) males were bred with mdx/utr (±) female mice to produce transgenic α7BX2 mdx/utr (−/−) mice.

### Example 3

### Tissue Collection and Storage

Muscle biopsies, for example from the vastus lateralis muscle, are obtained from dystrophic patients (or others of interest) and from normal humans using local anaesthetic. Irrelevant biopsy samples from the same patients and normal humans serve as controls. Biopsied muscle samples are frozen in liquid nitrogen immediately after removal. Further control muscle samples are obtained from normal individuals without any known muscle diseases. Muscle samples are stored at −80° C. prior to analysis.

### Example 4

### Antibodies and Reagents

For western blot analysis, the polyclonal antibody specific for α7CDA(345) and polyclonal antibody specific for α7CDB(347) are used to detect the α7A and α7B integrin cytoplasmic domains, respectively (Song et al., 1993). Peptides used to make these polyclonal antibodies were used as blocking controls. The monoclonal antibody O5 was used as a pan-α7 integrin probe. For immunofluorescence analysis the pan-α7 integrin monoclonal antibody O26 was used to detect all α7 integrin chains. Rabbit polyclonal antibodies to the cytoplasmic domains of the α7A and β1D integrin chains were provided by Dr. W. K. Song (See Kim et al., 1999, Cell Adhes. Commun 7:85-87). Dystrophin was detected using an anti-dystrophin monoclonal antibody (MANDRA1) purchased from Sigma Chemical Co., St. Louis, Mo. Culture fluid from the anti-utrophin monoclonal antibody-producing hybridoma (NCL-DRP2) was purchased from Novacastra Laboratories, Ltd. The anti-fetal myosin heavy light chain (fMYHC) monoclonal antibody 47A was obtained from Dr. Peter Merrifield (University of Western Ontario). AChR clusters were detected using rhodamine-labeled α-bungarotoxin purchased from Molecular Probes, Eugene, Oreg. FITC-labeled donkey anti-mouse and anti-rabbit antibodies were purchased from Jackson Laboratories, Bar Harbor, Me. The anti-creatine kinase monoclonal antibody (anti-CKIM) was obtained from ADI Diagnostics, Rexdale, Ontario.

### Example 5

### Western Analysis

Samples of muscle tissue were extracted in 200 mM octyl-β-D-glucopyranoside, 50 mM Tris HCl, pH 7.4, 2 mM phenylmethylsulfonyl fluoride, 1:200 dilution of Protease Cocktail Set III (Calbiochem, San Diego, Calif.), 1 mM CaCl₂, 1 mM MgCl₂ at 4 C for 1 hr. Particulate material was removed by centrifugation, and the supernatants were collected. Protein concentrations were determined according to Bradford, M. (1976) Anal. Biochem. 72:248-254. Equal amounts of extracted muscle proteins were separated by sodium dodecyl sulfate polyacrylamide (8%) gel electrophoresis at 40 mA for 50 min. The proteins were transferred to nitrocellulose filters. Filters were blocked using 10% horse serum in PCS, and the blocked filters were incubated with a 1:500 dilution of polyclonal anti-α7CDA(345) and anti-α7CDB(347) primary antibodies that recognize the A and B cytoplasmic domains, respectively (Song et al., 1993). Horseradish peroxidase (HRP)-linked anti-rabbit secondary antibody was used to detect bound primary antibody. Immunoreactive protein bands were detected using an Enhanced Chemiluminescence kit (Amersham, Arlington Heights, Ill.). Specificity of the bands was confirmed using the blocking peptides which served as immunogens in the production of the A2 (anti-α7A) and B2 (anti-α7B) antibody preparations. Blots were re-probed with an anti-creatine kinase antibody. The intensities of the α7 bands were compared to creatine kinase using ImageQuant software (Molecular Dynamics, Sunnyvale, Calif.).

### Example 6

### Immunofluorescence Analyses

Quadriceps muscles from 10 week old male mdx, mdx/utr (−/−) and α7BX2-mdx/utr (−/−) were embedded in OCT (polyvinyl alcohol and polyethylene glycol) compound (Tissue-Tek, Torrance, Calif.) and frozen in liquid nitrogen cooled isopentane. Using a Leica CM 1900 series cryostat, 10 μm sections were cut and placed on microscope slides coated with 1% gelatin, 0.05% chromium potassium sulfate. Sections were fixed in −20° C. acetone for 1 min, rehydrated in phosphate buffered saline (PBS) for 10 mm and blocked in PBS containing 10% horse serum for 15 min. The rat α7 chain was detected using 5 μg/ml of purified 026 monoclonal antibody directly labeled with Alexa 488 (Molecular Probes, Eugene, Oreg.). The anti-β1D antibody was used at a 1:100 dilution in 1% horse serum in PBS. The anti-dystrophin antibody was used at a 1:100 dilution while anti-utrophin and anti-fMyHC antibodies were diluted 1:2 in 1% horse serum, PBS. Rhodamine labeled α-bungarotoxin was used at a 1:3000 dilution to detect neuromuscular junctions.

Endogenous mouse immunoglobulin was blocked before the addition of monoclonal antibodies using 60 μg/ml goat anti-mouse monovalent Fabs (Jackson Laboratories,) in 1% horse serum in PBS, for 30 min at room temperature. Slides were then washed three times for 5 min each time in 1% horse serum in PBS. Primary antibodies were added for 1 hour at room temperature. Slides were washed 3 times (5 min per wash) in 1% horse serum, PBS. Primary antibodies were detected with a 1:100 dilution of FITC-labeled donkey anti-mouse or anti-rabbit antibody in 1% horse serum in PBS. Slides were mounted using Vectorshield mountant (Vector Labs, Burlingame, Calif.). Localization of the antibody was observed with a Zeiss Photomicroscope III (Carl Zeiss, Inc., Thornwood, N.Y.). Images of were acquired with a Sony DXC9000 color video CCD camera using SiteCam software (Sony, Tokyo, Japan).

Muscle biopsies from normal individuals and SPMD patients are embedded in OCT compound and frozen in liquid nitrogen cooled isopentane. Using a Leica CM1900 series cryostat, 10 μm sections were placed on microscope slides coated with 1% gelatin, 0.05% chromium potassium sulfate. Sections were fixed in ice cold acetone for 1 min, rehydrated in phosphate buffered saline (PBS) for 10 min and blocked in PBS containing 10% horse serum for 15 min. The α7 integrin protein was detected using 35 μg/ml purified O26 monoclonal antibody in 1% horse serum, 1×PBS. The anti-b1D antibody was used at a 1:100 dilution in 1% horse serum, 1×PBS. The anti-dystrophin antibody was used at a 1:100 dilution and anti-utrophin and anti-merosin antibodies were diluted 1:2 in 1% horse serum in 1×PBS. Rhodamine-labeled bungarotoxin was used at a 1:1000 dilution to detect neuromuscular junctions. After the addition of primary antibody, slides were incubated for 1 hr at room temperature in a humidified chamber. Slides were washed 3 time (5 min each) in 1% horse serum, 1×PBS. Primary monoclonal antibodies were detected using a 1:1000 dilution of FITC-labeled donkey anti-mouse or anti-rabbit antibody in 1% horse serum, 1×PBS. Washed slides were mounted in Vectorshield mountant (Vector Laboratories, Burlingame, Calif.) and coverslipped. Human α7 integrin protein bands were visualized using a Zeiss Photomicroscope III (Carl Zeiss, Inc., Thornwood, N.Y.). Images were acquired using a Sony DXC9000 color video CCD camera and Sitecam software.

### Example 7

### Histology

Ten micron cryosections from the quadriceps muscles of 5, 8 and 10 week old wildtype, mdx, mdx/utr (−/−) and transgenic mdx/utr (−/−) mice were placed on uncoated slides and stained with hematoxylin and eosin. The occurrence of central nuclei was scored in a minimum of 1000 fibers in two mice from each line.

### Example 8

### X-Ray and Magnetic Resonance Imaging

Spinal curvature (kyphosis) in 10 week old mdx, mdx/utr (−/−) and transgenic α7BX2-mdx/utr (−/−) mice was visualize by X-ray imaging using a Siemens Heliodent 70 X-ray machine (model D3104). X-rays were taken at 70 kVp and 7 mA.

Magnetic resonance imaging (MRI) of 10 week old wildtype, mdx, mdx/utr (−/−) and α7BX2-mdx/utr (−/−) mice was used to visualize soft tissues. Mice were imaged at 1 mm thickness using a 4.7T/3lcm Surrey Medical Imaging Spectrophotometer.

### Example 9

### RT-PCR and Genomic DNA Analyses

Total RNA was extracted from frozen muscle biopsies using TRIzol reagent (monophasic solution of phenol and guanidine isothiocyanate; U.S. Pat. No. 5,346,994; Gibco-BRL, Gaithersburg, Md.). A panel of overlapping primers designed from the α7 cDNA sequence were used in RT-PCR reactions to screen patient RNA for transcriptional expression of the integrin α7A subunit isoform. For example, in patients with scapuloperoneal muscular dystrophy, there is very little α7 integrin expression.

The primers used to amplify around the human α7A/α7B alternative splice site are hu3101F 5′-GAACAGCACCTTTCTGGAGG-3′ (SEQ ID NO:4) and hu3438R 5′-CCTTGAACTGCTGTCGGTCT-3′ (SEQ ID NO:5). In SPMD patients there is very little α7A amplification product in comparison to the amount seen in a normal individual. The expected product sizes from the use of these primers in a polymerase chain reaction are for α7A: 451 bp band; α7B: 338 bp band. The numbers in the primer names correspond to the location in the human cDNA sequence, F denotes a forward primer and R denotes a reverse primer.

For Southern hybridization analyses, mouse genomic DNA was isolated from whole blood or liver using a genomic DNA isolation kit (Promega). DNA was cleaved with EcoRI and Kpnl at 3 U/μg of DNA for 16 hours. DNA fragments were separated on 0.8% agarose gels and alkaline transferred to Hybond-XL nylon membranes (Amersham) (Sambrook et al., 1989). A 367 bp probe from the rat α7 3′-non-translated domain was isolated. The probe was directly labeled with HRP using a North2South non-radioactive kit (Pierce Scientific, Rockford, Ill.). The hybridized blots were washed following manufacturer's instructions. Probes were detected using an ECL substrate (luminol and H₂0₂, Amersham Life Science, Arlington Heights, Ill.). Blots were exposed to X-ray film from 1 to 30 min.

### Example 10

### Reporter Construction

TABLE 1

DNA sequence of a Portion of the Human α7 Integrin Promoter Region
(1970bp) (SEQ ID NO:6). The translation start site is underlined.

GAAAGTAGAATCCTGGTGCCAGCCCTGCTGACAGCATATGTATTTCCTTATAGTACCTGTTTAGAGATGTGTTAGTG
CTCTGGAGGGGATAGCCACAGGTGTAGTATTGGAAAACAGAGGGCCAGACTTCCAAATGTCTGTTAACTTATCCAAG
GCAAAGACTGTCCCAGGGCAGCAGAGTAAGAACCCACTTTTTTTTTGTTTTCAAAGAAGTATAATCCTGAACAATGA
AGTAGGAAAGACAGAACACAGGAAGAGGAAGGAGGTAGGACACTTATTGGAACTTTTAAGAAAGGGAAAGAGAAGAA
AGAATCGTAAGAATATGATAGTGTTTGAAGGGCAGAGACAACACTAGAAACATTGAGAAATACTCTGAGAAAGATTC
CAAGTGTGGCAGAGACAAGAATGATGACAAAATAGAATTTGGGATGAGACAAAATCAGATAGTGAGAGAGAGAAGGG
AAGATGGACAGATGTATATTCACAAGACCAACACCAGTAAGCAAGGGGAGTAGGAAGGGGAAGTGGGAGCATTCGAG
GTTCCCATTATGCCAAATTATTTCCTGTCTCTCCTTCTGGCCCCATTTCTGTATCGGAGTTATAAATAGCAGAGAGT
TGGAAAGTGTCCCCCCACCCCCTTGCCTCTGTCCCAGCCTGAGGGAAAGGGAGAGGAAGAGGGACAGGCCAATGGGT
CCCTGTGGAGATCCCATCTCAGCCCCACCCAGGTCCTGCTGAGCCAGTCCAGGACTCTGCCCCCTCCCATCCCCTTT
CATGGATAGGAAATGTGCAGTCCTGGGACGGGTCTGGTAGCTGGGGACACCCTTTACATCCCTCTGCCTCTTGGGTC
CAGTCTCTTTCATCTTTGCCTTCTTTGACACCCACTCCCCTCCCCACTGCTTAATTTCCTCTTCCTGTAATCATCCC
CAGTCGTTTTCTTTTCTCCGTTCATTCCATCCCTTGTCAATTAATCTCTTGCCCTTCTTTCTTCCTCTCTATTCCTT
TCCTTTTTCCATTTCTCCATTTGCTCCCCGTATCTCCCGAGTTTCTCTCTCTCTTCTTGCCTCTTTTTCTCTGTTCC
CTTGAATCCTGACGATGTGGCTAGCACTGCTGTGGTCATTGCCGGGCTGGGGGCGGGGGATGGGATAGGATGGGGGA
GGGCAGCGGTCTGATCCCAACAGCAGAAAGAGTGCTCTATGTGACCATGGGGGAACAGGGAGCACTAAGATGCCACG
CTGCACCCAGGCCCAGGACGGCTCCCCTTTCATTTCCTCTCTATCTGCACATCTCTCTTCCCAGGTTGTCTTTTAGC
GTCTTCCCAACTTCTCATCTCTTACCCTCCTTCCTCTGTTTCAGCCCCTCTCTTTCTATCTGTACTTCTCTCCCTCC
GCATTCCAAGGCGCCGCCTCCACCACTCCCGGGGTGGGGATGGGGTTGGGGGAGAAGGGGAGGAGAGCGCCGCGCAG
GGGCGGAGCCGGAGACGGTGCTGGGCTTGGGGGGCGTGGTGGTGGGGGGTCAGCAAGGCTAGTTTCCATCCCAGCCA
CCAGCCTGGGCATCCCCTTGGAGACGGGCTTGGGTCTCCACCTGCCGCGGGAGCGAGGGGCGGGGCCGGAGGCGGGG
CCTGAGTGGCGTCCCCGGGAGAGGAGGCGGGAGCCGGAGTGGGCGCCGGAGCTGCGGCTGCTGTAGTTGTCCTAGCC
GGTGCTGGGGCGGCGGGGTGGCGGAGCGGCGGGCGGGCGGGAGGGCTGGCGGCGCGAACGTCTGGGAGACGTCTGAA
AGACCAACGAGACTTTGGAGACCAGAGACGCGCCTGGGGGGACCTGGGGCTTGGGGCGTGCGAGATTTCCCTTGCAT
TCGCTGGGAGCTCGCGCAGGGATCGTCCCATGGCCGGGGCTCGGAGCCGCGACCCTTGGGGGGCCTCCCGGATTTGC
TACCTTTTTGGCTCCCTGCTCGTCGAACTGCTCTTCTCACGGGCT

A luciferase reporter system is used to analyze promoter activity and to identify compounds which modulate (increase or decrease) α7 integrin promoter activity. The isolated α7 integrin promoter sequences are subcloned into the pA3Luc vector so that the firefly luciferase gene is under the transcriptional control of the human α7 integrin promoter. These constructs are transfected into a human myoblast cell line along with a control vector phRL-TK(lnt-) containing the Renilla luciferase gene coding sequence. Cotransfection with the Renilla construct is used to control transfection efficiency. The different fragments of the human α7 gene are analyzed to determine which contains the greatest activity as determined by the luciferase reporter. The fragment with maximum activity is subcloned into the β-lactamase reporter system for subsequent screens. In addition to the approximately 2 kb transcriptional regulatory sequences disclosed herein, an approximately 5 kb fragment of the human α7 integrin promoter is also useful in reporter gene constructs. Alternatively, a sequence of about 2.8 kb can be used as described by Ziober et al. (1996), incorporated by reference herein. Another reporter system useful in the context of the present is the GeneBLAzer β-lactamase reporter technology (Aurora Biosciences Corporation, San Diego, Calif.).

The reporter gene constructs of the present invention are transformed into myoblasts or myotendinous cells. Desirably, the reporter sequences are recombined into the chromosome at the α7 locus such that the reporter is expressed under the regulatory control of the native α7 promoter and associated regulatory sequences. These cells in which the reporter gene vector is maintained (or incorporated into the genome) are contacted with test compounds, and the effect on reporter gene expression is monitored (fluorescence intensity where the reporter gene coding sequence is that of a fluorescent protein such as aequorin) and by measurement of a detectable product of an enzyme coding sequence, e.g. and enzyme activity such as that of β-lactamase in the case of the GeneBLAzer system or that of luciferase using the reporter vector described above. Those compounds which cause a higher level of reporter activity in the presence of than in the absence of the compound are those which stimulate expression of the intact α7 integrin. These compounds similarly increase the level of α7 integrin in muscle and myotendinous cells. As demonstrated herein, increased expression leads to an amelioration of the muscular dystrophy symptoms.

The human α7 integrin transcription regulatory sequences are identified as part of the Homo sapiens chromosome 12 BAC, RP11-644F5. This BAC nucleotide sequence is available under GenBank Accession No. AC009779. A useful α7 integrin transcription regulatory sequence of about 4 kb is from nucleotides 25,511 to 29,515 and a useful non-regulatory sequence of about 5 kb is from nucleotides 32,639 to 37,599.

The reporter gene vector is useful to produce stable transfectants of human myoblasts, which can then be used to screen for compounds (or conditions) that increase integrin expression in skeletal muscle. Having the endogenous ITGA7 promoter linked to a reporter gene provides the most natural target with which to screen for compounds (or conditions) that alter integrin expression.

A 4 kb fragment comprising the ITGA7 promoter, including the transcription regulatory sequences, is cloned 5′ to the luciferase gene. This construct is called pGL3-ITGA7-4 kb. Good expression of the luciferase reporter is seen when transfected into human PC-1 cells and mouse C2 myoblasts. Next, the 4 kb fragment and luciferase gene were PCR-amplified using primers incorporating a Not I restriction site. Forward: TCAGTTTCTCAGTCATACTAGCC (SEQ ID NO:7; REVERSE: AATATTTAGCGGCCGCGGGCATCGGTCGACGGATC (SEQ ID NO:8). This 4 kb Luc fragment was cloned upstream of the Neo gene in the vector pTKLNCL (Mortensen et al. (1992) Mol. Cell. Biol. 12:2391-2395). Next, the ITGA7-5 kb fragment was PCR-amplified from the BAC RP11-644F5 using primers FORWARD: TATCTCGAGCTTATATCCCTGGTGTCTAGCC (SEQ ID NO:9); REVERSE: TATCTCGAGTTAATTAATCTCATTCCACCTGAATCTTCC (SEQ ID NO:10), which incorporate a Xho-I restriction site. This 5 kb fragment is cloned downstream of the cytosine deaminase (CD) gene in the above construct. The Lox P sites in the pTKLNCL vector are used to remove the Neo gene from the stably transfected cells. pGL3-ITGA7-neo-α7intron-TK linearized with Pac1 is transfected into human PC-1 myoblasts (or mouse C2 myoblasts) (Arbones, L. et al., 1994. Nature Genetics 6:90-97) and neomycin resistance is selected. The CD gene ensures that in the presence of 5-fluorocytosine cells retaining the Neo gene are killed. Cells selected for growth in the presence of neomycin are then grown in the nucleoside analog gancyclovir to select for stable homologous recombinants without the TK gene. The strategy for directing homologous recombination is shown in FIG. 9A.

Random insertions of the plasmid sequences are likely to contain the TK gene, and these can be selected against using ganciclovir.

Recombinant myoblasts, preferably human, carrying the reporter vector are treated with test compositions in parallel with a buffer or solvent control. Reporter activity in the paired samples is compared; compositions that increase reporter activity at least 30% over the control are deemed to be positive modulators of integrin expression. Those which lower reporter activity 25% or greater are deemed to be negative modulators of integrin gene expression.

### Example 11

### Statistical Analysis

Survival data from 84 mdx/utr (−/−) mice and 43 transgenic α7BX2-mdx/utr (−/−) mice were analyzed using the Kaplan-Meier method (Kaplan and Meier, 1958). Survival curves were generated for both populations and the data compared using log-rank (Peto et al., 1977) and Wilcoxon (Conover et al., 1980) statistical tests.

TABLE 2
Percent fibers with central nuclei

| | 5 weeks | 8 weeks | 10 weeks |
|---|---|---|---|
| Wt | 2.6 | 1.3 | 2.7 |
| Mdx | 33.0 | 65.6 | 70.9 |
| mdx/utr (—/—) | 79.0 | 78.4 | 75.2 |
| α7BX2-mdx/utr (—/—) | 62.1 | 71.7 | 63.9 |

i. Sections of hindlimb muscle from 5, 8, and 10 week old mice were
ii. stained with hematoxylin and eosin. Nuclear localization was scored

iii. in at least 1000 fibers in each animal.

TABLE 3

Excerpted Nucleotide Sequence from NCBI Accession No. AC009779 (Human
BAC12). See also SEQ ID NO:7, which corresponds to this excerpt from
the BAC sequence. Nucleotides 25,511 to 29,515 and nucleotides 32,649
to 37,599 of the BAC12 sequence correspond to nucleotides 491 to 4495
and 7629 to 12579, respectively, of SEQ ID NO:11.

| | | | | | | |
|---|---|---|---|---|---|---|
| 25021 | ccactgaatt | ccctcaatca | catttatgtt | cttttcctcc | cagccactcc | catggttcaa |
| 25081 | gctttgacta | caaccagaat | tcagaggcag | gcagaaggat | tccagtgctg | gagaggtgag |
| 25141 | tgaagtaaaa | aagttctcat | ggtgtgcatg | ttgggacgga | aaagcctgac | cttgggacat |
| 25201 | aagctccaag | gctctgttgc | cagatgaggt | ggagggagaa | gttagccctg | aagtgtgtgt |
| 25261 | tctggaagtg | tttgcttgta | agctagagac | aacagttgca | aaaagtgtga | tttgagggag |
| 25321 | ctgaaaaata | ctgatctcaa | agtggggaag | aagatgttga | aaagggaagg | agctggagaa |
| 25381 | agcctcagct | tccactcata | caaaagctaa | agggctaaaa | tcttggctgg | atctggacat |
| 25441 | ttctcaacgt | ctaaaatttt | ggaaattttt | ataaagatta | ttaatctttc | atttttacat |
| 25501 | ttaatttatt | taaaaagttc | agtttctcag | tcatactagc | cacatttctt | tttctttttc |
| 25561 | tttttttttg | agacagagtc | tcactctgtt | gcccaggctg | aagtacagtg | tattaatcta |
| 25621 | ttccatggag | tggagtggat | aatctattcc | atggattatc | attttacttt | gttagtggta |
| 25681 | tccttagaag | cacaaaattt | ttaaattttt | tttttttttt | gaggcagagt | ctcactgtgt |
| 25741 | cccccaggct | ggagtgcagt | ggtgctatct | ctgctcactg | caggctccgc | cttctgggtt |
| 25801 | caagcgattc | ttgtgcctca | gcctcctgag | tagctgggat | tacaggtgtg | taccaccacg |
| 25861 | cccagctgat | ttttgtattt | ttagtagaga | tgggtttttg | tcatattggc | caggctggtc |
| 25921 | tcaaactcct | gacctcaagt | gatccacctc | cctccctccc | aaagtgctgg | gattaccatg |
| 25981 | tctggcttgg | aaattatttt | gaaataatta | tagatcagag | gaagttgtaa | aaatagcaca |
| 26041 | tgaagtcttg | tgtacctttc | actcagtttc | ccctaatggt | gacatcttat | gtaactgtag |
| 26101 | cataaaatca | aaaccaaaaa | gttgatattg | gtacagtatt | gttaactggc | ctgcagacct |
| 26161 | cactcagttt | tcaccatttt | ttacatgcat | ttatttgttt | gtttgtagtt | ctgtgcagtt |
| 26221 | ttatatcttg | tatagatttg | gataatcacc | accacaatca | agatacaaaa | cccatcacca |
| 26281 | caaaggaacc | cccttgtgct | attcctttat | gtttgtcccc | acccccctcc | atccttgtcc |
| 26341 | cctggcagcc | agtaatctgg | tcttcatttc | tatagttttg | tcattttgag | aatggtatgc |
| 26401 | gagtggaata | atacagtttc | agcatttttt | gtttggagac | agggtctcac | tctatcaccc |
| 26461 | aggctggagt | gcagtggcaa | gatcatggct | cactgcagcc | ttcacctcct | gggctcaagt |
| 26521 | gacactcccg | cctagcctcc | tgagtagctg | ggaccacaga | tttggctaac | ttttctattt |
| 26581 | tttgtagaga | tgggggtctc | cctatgttgc | ccaggctggt | ctccaactcc | tgggctcaag |
| 26641 | tgatcctcct | gccttggcct | ctcaaagtgc | tgggattgca | ggcatgagcc | actgtgccca |
| 26701 | gctcagcatt | aatttttaat | ttaactaatt | cctaagctct | tgactgaaat | acaagaagtt |
| 26761 | ctctaacagt | ttatttattt | taatattgag | cttaccgcat | tctctggatc | cttctagttt |
| 26821 | cttttttttt | tctttttttc | tgatgtggag | tctctgtcac | ccaggctgga | gtgcagtggt |
| 26881 | gccatctcag | ctcactgcaa | cctccgtctc | ctgggtttaa | gtgattcttg | tgcctcagtc |
| 26941 | tctggagtag | ctgggattac | aggtacccgc | caccacaccc | ggctaatttt | tgtattttta |
| 27001 | gtagagacag | ggtttcaccg | tgttggtccg | gctggtcttg | aacttctgat | ctcaggtgat |
| 27061 | ccgcctgctt | cggcctccca | aagtgctggg | attataggcg | tgagccaccg | cgcccggccc |
| 27121 | cgtctagttt | cttaatttcc | ctcttcacct | acgatattat | cttccactcc | aacattctgg |
| 27181 | tctcatttct | ccttgagaga | aatctacatg | tctaaattta | ctaggctggt | ctagcacgct |
| 27241 | cttgtgtgtt | cccctccctc | ctttgcccct | ctatttatag | ccaggctaat | tttgggtggc |
| 27301 | ctctctctct | cttctttcct | gatctttcct | cctgtggtgg | tgaggtgact | tctcaaatat |
| 27361 | ttggagagag | gaggtcagaa | gcagattctt | ggcatctgat | ttcagccctg | gatcacagaa |
| 27421 | gccagtggag | tgggaatgga | gacaggcaga | agctgcaggt | gcagatagga | ggcagcttgg |
| 27481 | gctctaaagg | cattttgagc | tgggtcgggg | gcggggggac | ctgggcaggg | agtcagtagt |
| 27541 | cccagttctg | tcctaatttt | gcaattctgc | attcccatgt | cagctcttct | ctactgtctg |
| 27601 | gggctctgag | atattaaaaa | ggatggggag | ggcatggtga | aagtagaatc | ctggtgccag |
| 27661 | ccctgctgac | agcatatgta | tttccttata | gtacctgttt | agagatgtgt | tagtgctctg |
| 27721 | gaggggatag | ccacaggtgt | agtattggaa | aacagagggc | cagacttcca | aatgtctgtt |
| 27781 | aacttatcca | aggcaaagac | tgtcccaggg | cagcagagta | agaacccact | ttttttttgt |
| 27841 | tttcaaagaa | gtataatcct | gaacaatgaa | gtaggaaaga | cagaacacag | gaagaggaag |
| 27901 | gaggtaggac | acttattgga | acttttaaga | aagggaaaga | gaagaaagaa | tcgtaagaat |
| 27961 | atgatagtgt | ttgaagggca | gagacaacac | tagaaacatt | gagaaatact | ctgagaaaga |
| 28021 | ttccaagtgt | ggcagagaca | agaatgatga | caaaatagaa | tttgggatga | gacaaaatca |
| 28081 | gatagtgaga | gagagaaggg | aagatggaca | gatgtatatt | cacaagacca | acaccagtaa |
| 28141 | gcaaggggag | taggaagggg | aagtgggagc | attcgaggtt | cccattatgc | caaattattt |
| 28201 | cctgtctctc | cttctggccc | catttctgta | tcggagttat | aaatagcaga | gagttggaaa |
| 28261 | gtgtcccccc | acccccttgc | ctctgtccca | gcctgaggga | aagggagagg | aagagggaca |
| 28321 | ggccaatggg | tccctgtgga | gatcccatct | cagccccacc | caggtcctgc | tgagccagtc |
| 28381 | caggactctg | ccccctccca | tcccctttca | tggataggaa | atgtgcagtc | ctgggacggg |
| 28441 | tctggtagct | ggggacaccc | tttacatccc | tctgcctctt | gggtccagtc | tctttcatct |
| 28501 | ttgccttctt | tgacacccac | tcccctcccc | actgcttaat | ttcctcttcc | tgtaatcatc |
| 28561 | cccagtcgtt | ttcttttctc | ccttcattcc | atcccttgtc | aattaatctc | ttgcccttct |
| 28621 | ttcttcctct | ctattccttt | cctttttcca | tttctccatt | tgctccccgt | atctcccgag |
| 28681 | tttctctctc | tcttcttgcc | tctttttctc | tgttcccttg | aatcctgacg | atgtggctag |
| 28741 | cactgctgtg | gtcattgccg | ggctgggggc | gggggatggg | ataggatggg | ggagggcagc |
| 28801 | ggtctgatcc | caacagcaga | aagagtgctc | tatgtgacca | tgggggaaca | gggagcacta |
| 28861 | agatgccacg | ctgcacccag | gcccaggacg | gctccccttt | catttcctct | ctatctgcac |
| 28921 | atctctcttc | ccaggttgtc | ttttagcgtc | ttcccaactt | ctcatctctt | accctccttc |
| 28981 | ctctgtttca | gcccctctct | ttctatctgt | acttctctcc | ctccgcattc | caaggcgccg |
| 29041 | cctccaccac | tcccggggtg | gggatggggt | tgggggagaa | ggggaggaga | gcgccgcgca |
| 29101 | ggggcggagc | cggagacggt | gctgggcttg | gggggcgtgg | tggtgggggg | tcagcaaggc |
| 29161 | tagtttccat | cccagccacc | agcctgggca | tccccttgga | gacgggcttg | ggtctccacc |
| 29221 | tgccgcggga | gcgaggggcg | gggccggagg | cggggcctga | gtggcgtccc | cgggagagga |
| 29281 | ggcgggagcc | ggagtgggcg | ccggagctgc | ggctgctgta | gttgtcctag | ccggtgctgg |
| 29341 | ggcggcgggg | tggcggagcg | gcgggcgggc | gggagggctg | gcggggcgaa | cgtctgggag |
| 29401 | acgtctgaaa | gaccaacgag | actttggaga | ccagagacgc | gcctgggggg | acctggggct |
| 29461 | tggggcgtgc | gagatttccc | ttgcattcgc | tgggagctcg | cgcagggatc | gtcccatggc |
| 29521 | cggggctcgg | agccgcgacc | cttggggggc | ctccgggatt | tgctaccttt | ttggctccct |
| 29581 | gctcgtcgaa | ctgctcttct | cacgggctgt | cgccttcaat | ctggacgtga | tgggtgcctt |
| 29641 | gcgcaaggag | ggcgagccag | gcagcctctt | cggcttctct | gtggccctgc | accggcagtt |
| 29701 | gcagccccga | ccccagagct | ggtgagtcac | cgcacccgcc | cagagtcgcc | atgcccgagc |
| 29761 | cacagatcgt | ccccctcccc | actctgtggg | cctcctcatt | tctctgtttt | ctagccccac |
| 29821 | caagacctag | actgcccaca | gacatcccac | atcccaacct | ggagccttgc | ctcatctggc |
| 29881 | ttgcgtctga | agctgcactt | cccggccctg | agaccagtat | tttgctttag | ggatgagttg |
| 29941 | gaaagcaagg | ttcttgtctt | ggcagcgaac | catctccttc | ttctgggcct | ttcccccaac |
| 30001 | ttgcatcctt | gatccagccc | cagggcctct | ggctcccctg | cttcttccaa | gggctgaatt |
| 30061 | ccccaaggga | gggagactgt | ctgtctctgc | ttagaatggg | aggagatgga | aaggacatag |
| 30121 | aagttgaggg | tgccatgaga | gggatgcatg | cagggcagac | tccagaaata | acttcctgct |
| 30181 | agagcattgc | catggatgga | atgagggcag | cagggcactg | gaaggccagg | agagagcttc |
| 30241 | cacttctgtg | gcttaagacc | acgggaagat | tgggagagga | tctgcaggtc | tgccaacctg |
| 30301 | cagtaggtgg | cttggtgata | gagagtggca | gcaaactgaa | ccctcaaagt | actagtagca |
| 30361 | gtagtagtag | ccgcagcagc | tgtagcagtg | agagagatcc | aggaaggatg | ctggccaggc |
| 30421 | tgctcccctt | cctcctcctt | agcaaatttc | caactccagg | aatctcagca | gctgggaagg |
| 30481 | gccaggagga | gtaaggggtg | gaggacaatt | ctaatttttt | ctaatcagtt | caggacccat |
| 30541 | gggagatgga | tatacttttg | tgaggggcct | gtgactggtc | atgttgcctg | tatccttggc |
| 30601 | tcttgctaca | tgtctgattg | taaaaaggga | ggccagaggt | gaagaaagct | tctcacctgc |
| 30661 | tcctgctagg | gggcttttct | ctcttcaacc | agtgcctaag | ccacattaag | tatccattac |
| 30721 | tgggatcaat | gctgtccact | gggactgtct | tctgcctcta | ctgtcggtct | gggggcaggg |
| 30781 | ggcagggaca | agagctcatt | tctcctcact | tgcttgggga | gtgggggcct | agctctaatc |
| 30841 | tttcttcttc | cattatccct | atcatctggt | agcagggtcg | gtggtcccca | aaactttggg |
| 30901 | agagatagaa | agcaacggac | ttcatctcct | cttctgttta | ccatctgctt | cctcattcac |
| 30961 | ctttgctccc | tccctccctt | cctccctcct | tctccatctg | tcagagttcg | aggactggag |
| 31021 | gcctttttag | gacatgctga | actctctaag | ctatttccag | gcaaattcta | ggttattttt |
| 31081 | aatagcttgg | tctcttgtca | tttccccctc | ctctctgaag | gtggcccctg | gttccgtctc |
| 31141 | ccagagccaa | gctggggcct | ttcccagagg | gcctgactgc | ctcaccctgc | ttttgctcca |
| 31201 | gcagggggtg | ctctgctgct | ggggggcggg | gggtatgtga | gaggccaggc | acctgctcag |
| 31261 | tccctagctt | ttgagttgca | ggtggcctgc | cttagcactc | actgatgaaa | aaaacttctt |
| 31321 | gcctgttttg | atgtctttta | gtctagctct | gggatgagac | tttaaggtct | aacctttgct |
| 31381 | gtgtggttcc | agcctcattt | acttccctca | actgtaaaaa | ggatataaac | atagtattac |
| 31441 | tacatagggt | tgttgtgcag | attaagagtt | cttaatatat | ataaaatgct | tagaatagtg |
| 31501 | catagcccgc | agtgagtgct | gtgaagtgtt | agtagtattg | ctattcttgt | attgtgattc |
| 31561 | acagagcgcc | ttacagagat | tctggatcca | aaggcttggc | tagagggcct | ccctggctga |
| 31621 | gccagccttc | caggccaagc | atcctcccca | gagggccacc | cagattgaga | ggggccaaag |
| 31681 | aggggctgga | cttgggctgg | ggccctggag | tgtgtggaga | atcgagaagt | gcagtggtcg |
| 31741 | tgggctactc | ctttgtcttc | acttagctga | gctcccaggg | ggtccctctg | ccccccagct |
| 31801 | gccaacactt | tttttttttt | ttttttgctt | ttctctctgc | agtggctaca | ctgtggctgt |
| 31861 | ccagaagact | ggggtggtta | gggcgtatgg | catgaagcca | ggaaggagtg | tgtgtggctg |
| 31921 | gaccagaggt | ggagggacta | gagaggatgc | tgctgggtgc | tcttgttcca | ctaaggatcg |
| 31981 | attggtctct | tctccaccaa | gagcggactg | ggcatatcta | tgcactcagc | ttctttcttc |
| 32041 | cacatgggcc | cctcccctcc | tccctacttt | tggcctccag | aggagatgtg | aacatagaac |
| 32101 | aaggataact | tatctgggtg | cttagctatg | cactgaccag | ctgtgacact | gggtatctct |
| 32161 | atgagtccac | aaaatgtgtg | tgttcagtaa | acacattctg | acactcccta | tggggcaagc |
| 32221 | acaaagatga | aaagacagcc | ccgacactca | gagagatggc | cacttctatg | tttggaggct |
| 32281 | gggggtactg | ctgacttgcc | tgaaggttgc | catttattta | tgcagggctg | tatcaccccg |
| 32341 | tttccttttc | tgcccagggt | accctcatct | ccccactctc | tccttccctt | tctggggtgg |
| 32401 | tctcagtgtt | ctagagacag | gtcagtcact | gggtggagtg | acaaagtgtt | ggagttaggc |
| 32461 | ccatgtggat | ttgaattcca | gcatcactgc | ttaatgtctt | tgagtgagtt | ttctcatctg |
| 32521 | aaagacaaga | aaagaatcct | tatctcatag | gattgttctg | atgattaaat | gacataatgc |
| 32581 | atgtgacttg | cctatcctgg | tgcttggcac | atatgtggac | agtgatgaat | gttagtttct |
| 32641 | tatatccctg | gtgtctagcc | tcggatctga | cgtcatagta | ggtgctgaat | aaatatgatt |
| 32701 | tccttgtctc | accagcgcct | gacacagggc | ttggcataca | atagactctc | aataagtagt |
| 32761 | tgaatgccaa | atgtgtcttc | tcttctctac | tactccctat | accccttctc | tgtcttgaca |
| 32821 | ctggctctga | caagggatgg | cagctgctaa | gagatgagga | ggagttgtgg | gaaggaagaa |
| 32881 | tggctctctg | ccctccccct | ccaccccatc | agagctggca | cagtgcccca | cagatgcctg |
| 32941 | tctgtaatac | tgcctaacat | ggttttgggc | cttgcccccc | aggaagggag | atggaggaga |
| 33001 | agagtgtggg | agagaggcgt | tgaggtttgt | cccactgcca | cttctgagtc | tctccttctg |
| 33061 | caaagagagg | acccatggag | ccagctgggt | gtcagtcatc | ttacctcacc | cccgccttca |
| 33121 | ctctggcttg | ggggttcagc | cccaggggac | ccaggcagcc | tccattccca | gcactgtgct |
| 33181 | cccctgggga | agacggcttg | gctgtgatca | tggaaaattg | tcctgccaag | aaagttgtag |
| 33241 | ctgggaaaga | ggctgagggg | gaggcaggag | agaagactgg | gtgggggtgg | aagggaagga |
| 33301 | gaaatcatgg | acatggggag | aaggaaggat | ggggaagggg | attcaggatg | tcgggaagag |
| 33361 | aatggggtag | cattggaggc | agaaggagaa | acttgtccct | acctccatgc | cagccagagt |
| 33421 | gactgatgga | atcctgggct | ggcacagctt | ctgggaggtg | gggtctttgc | tgggtccctg |
| 33481 | atgagggggc | agtgggtccg | tatctagcct | cttgcctggc | ctctgaagct | ggtccctgag |
| 33541 | ccacactctg | atgccagtct | ggggccctgt | tacttttgct | cccagcattc | ttggcatttc |
| 33601 | tggctgggtt | tcaactggac | tgggttgggg | agcagggcag | agcttgggga | tggggccaag |
| 33661 | gaggggatag | ggaaggccta | ctcaggaaca | ggtgctggga | acaggcagtt | ctttcaaacc |
| 33721 | agcactgttg | gcctggctgc | ttgggttggc | gtgtatgtgt | gtgtgtgtgt | gtgtgtgtgt |
| 33781 | atctactgtg | tatgttgatc | ccttatccag | atagtatgta | catgcaacgt | gatgactgca |
| 33841 | tgaccaagca | tattaatttg | tccttgccag | ggtttgagaa | aactgacatt | tgccccttct |
| 33901 | ctttagtcct | tgaacactct | ctttagtact | gaggggttgg | gcctgggcag | ctctaatgag |
| 33961 | attgggtcat | tctgacctct | aactcctgtc | cctgtccctg | cccctgcccc | atcttgcagg |
| 34021 | ctgctggtgg | gtgctcccca | ggccctggct | cttcctgggc | agcaggcgaa | tcgcactgga |
| 34081 | ggcctcttcg | cttgcccgtt | gagcctggag | gagactgact | gctacagagt | ggacatcgac |
| 34141 | cagggaggtg | tggccctgca | tgaacagagt | gggggaagcg | tgtgagcggg | gaggagagga |
| 34201 | cttgggctcc | tcttccctcc | cctaattccc | agtgtcctgc | ctctagctga | tatgcaaaag |
| 34261 | gaaagcaagg | agaaccagtg | gttgggagtc | agtgttcgga | gccaggggcc | tgggggcaag |
| 34321 | attgttgtga | gtattgcttc | tcatgactga | atgcacggat | ggggtgtgtg | tgtgtgtgtg |
| 34381 | tttatggtgt | gtgcatacgc | ataggtgtgc | ttagagaaca | caagttagga | atatggtatg |
| 34441 | attccaagta | catcagggag | atataaaaag | gtgtgagaca | tggtccttgt | ccttataaat |
| 34501 | gtaaaaatgt | ctgtccattc | attcatccat | ccatttgtca | aactcttact | gagaaccttt |
| 34561 | taagcatcag | gcattgtgct | agttactaca | ggggaaggct | catgcctgta | atcccagcac |
| 34621 | tttcggaggc | cgaggcaggt | ggatcacctg | aggtcaggag | ttcgagacca | gccggaccaa |
| 34681 | catggcgaaa | ccctgtctct | cctaaaaata | caaaaaaatt | agccgggcgt | ggccgggtgc |
| 34741 | ggtggctcac | gcttgtaatc | ccagcacttt | gggaggccga | ggtgggtgga | tcacgaggta |
| 34801 | aggagatcga | gaccatggtg | aaaccccgtc | tctactaaaa | acacaaaaaa | ttagccgggc |
| 34861 | gtggtggcgg | gcgcctgtag | tcccagctac | tcagagaggc | tgaggcagga | gaatggcgtg |
| 34921 | aaccggagag | gcggagcttg | cagtgagctg | agatcgcgcc | actgcgctcc | agcctgggtg |
| 34981 | acagagcgag | actccgtctc | aaaaaaaaaa | aaaaattagc | taggtgtggt | ggcaggcgcc |
| 35041 | tgtaatccca | ggtactcggg | aggctgaggt | aggagaatca | cttgaacctg | ggtggaggag |
| 35101 | gttgcagtga | gctgagatcg | caccattgta | ccctagcctg | ggagacaaga | gcaaagttcc |
| 35161 | gtctcaaaac | caaccaaaca | aacaaacgaa | aaaaccagag | ctctctgttt | ctctctctct |
| 35221 | ctctatcttt | cagtaacacg | catagataca | caattaccaa | tacagatcac | tgtggggcag |
| 35281 | aatctggttc | atgttaagtg | agtggtctag | tctccagtct | ataaaagtcc | aaaggaggag |
| 35341 | tagagagaag | acttctgcag | aggggatgat | ttgagccagg | ctttaataat | aggtaatacc |
| 35401 | tagcctgtgc | aacatagtgg | gacctcatct | ttataaaaaa | taaaaacaaa | ttagccagtc |
| 35461 | atggtggtgc | atgcctgtag | tcccagctac | acaggaggct | gaggtgggag | gatcacttga |
| 35521 | gccctggagg | tcgaggctgc | agtgagccat | gattgtgcca | ctgcactcca | gcctgggtga |
| 35581 | cagagtgaga | ccttgcttca | aaaaaaaaaa | aaaagtaata | cttggagagt | gaagcggaca |
| 35641 | ggaagttctt | tgcagatgag | atggtgacac | ttacaaaggt | ccagggacag | ggccaagctt |
| 35701 | ggcattttgg | aggactgtga | catgatcagg | gagacacaca | tcctatgtgg | tggcttaatt |
| 35761 | gtgtcttttg | gctccaggca | gaatgtggaa | caaggagatc | tccatttgag | ggcaaggaag |
| 35821 | tgggtgcaga | caggttgctg | ggttatgcat | ggacctgtgt | aacactggca | gggtaatggt |
| 35881 | gcttgagtgg | tgccggcata | ggggtgtgtg | tgtatgtgtg | catgtgcatg | tgcatgtgag |
| 35941 | cacacatgta | tcagtatctg | ccaaatctct | gcatatgggc | agcatgcctc | aagcaggtcc |
| 36001 | ctggcccaca | gagtgaaatg | atccccatcc | cttcctcccc | cagacctgtg | cacaccgata |
| 36061 | tgaggcaagg | cagcgagtgg | accagatcct | ggagacgcgg | gatatgattg | gtcgctgctt |
| 36121 | tgtgctcagc | caggacctgg | ccatccggga | tgagttggat | ggtggggaat | ggaagttctg |
| 36181 | tgagggacgc | ccccaaggcc | atgaacaatt | tgggttctgc | cagcagggca | cagctgccgc |
| 36241 | cttctcccct | gatagccact | acctcctctt | tggggcccca | ggaacctata | attggaaggg |
| 36301 | tgagtcactc | ctcgggaagg | ggagaagggg | accaaaacct | cctcttacct | cagagacagg |
| 36361 | gttggggatg | gcacatggcc | aagcatgacc | acatgtgcac | tgctgtatgg | ccccagggca |
| 36421 | ctgccatgcc | ttccacccca | ttgagctagt | gcacacatga | atggggggtg | cctcctttcc |
| 36481 | ctcgcacggc | caagtgttcc | tcaacatgct | ggcatgggcc | ccaagtgcac | gctgggcctg |
| 36541 | cagctggggc | ctgcatgctc | caacacacta | gcccacacct | catcactgcc | attcccgtct |
| 36601 | ccgcacgctg | ctgctggctg | agctgacact | cggtgagtgt | gatgccacat | ctgggggacc |
| 36661 | ccaggaagcc | tgggttgggg | acagggtggg | gagagggcta | gaaagaagag | gcagggcttc |
| 36721 | cccgtgtgcc | tgtctaactc | agtgtccggc | ctgaggggtg | ttccttgcgc | cctgccctgg |
| 36781 | gcactaacag | gtctgtcctt | gcaggcacgg | ccagggtgga | gctctgtgca | cagggctcag |
| 36841 | cggacctggc | acacctggac | gacggtccct | acgaggcggg | gggagagaag | gagcaggacc |
| 36901 | cccgcctcat | cccggtccct | gccaacagct | actttggtag | ggacctctcc | ccggcccaga |
| 36961 | actgctctaa | ccctctgctc | ctctctcttg | tcctctctct | ccatgctccc | atccttctgt |
| 37021 | ctctgtttct | gtctctcacc | ttgtctctct | ctgtctttct | gtctctggct | gtgatctctc |
| 37081 | tggtctcttt | ttctctctcc | acctcttctt | cttccaccat | tttctggcct | ttctgtggct |
| 37141 | ctgtctccct | actctgtggc | ccctactctg | gatgtcccct | ccctggtgtc | tcaccccacc |
| 37201 | ccccacaggg | ttgctttttg | tgaccaacat | tgatagctca | gaccccgacc | agctggtgta |
| 37261 | taaaactttg | gaccctgctg | accggctccc | aggaccagcc | ggagacttgg | ccctcaatag |
| 37321 | ctacttaggt | ttgtaagctc | ccacctcctg | gactctaggg | gcatggccca | gcctcccctc |
| 37381 | cttccccagg | gaactcgacc | tttggtgcct | tataatctcc | tcctccccca | acacacaccc |
| 37441 | agggagacat | acattgggcc | caaattgcag | agaagagctg | ggtccaatga | tcaggcctaa |
| 37501 | gaggaggagg | cccccagggt | ggtggcctct | ggggctgtga | gccaggggtc | tccatggagg |
| 37561 | aagattcagg | tggaatgaga | gggccagggc | tgaggatatt | ttgggaagga | cagtcctgtc |
| 37621 | ttctaggggg | actttccctg | aggggatgga | tggtgggcac | atattgaaga | aagggctaat |
| 37681 | gttgttggta | agtccctctc | gttgtctcat | ctgcattcct | ctgcagagga | ggaggaaacc |
| 37741 | aggcctggga | gatgtttggg | tgaagcaggc | gctctctcac | tcccccttgt | ctccccctca |
| 37801 | tccatgtgaa | gacttcccct | ccctgccagg | atgagggagt | tgggggaaag | aggtgcactg |
| 37861 | ggtgggattc | gggcctgaga | gggacctcta | gctcttctag | ctccctgggt | gtgggcaggg |
| 37921 | tgaggccact | gtgctcagcc | tcctacctgg | gctcctggcc | ttctcagcca | tcacctttct |
| 37981 | ctctcttgcc | cagtccctga | ggctgacctc | actgcacttt | ttgtgccaag | cttgtctctg |
| 38041 | ggcctggtgg | gtgtgggagg | ctgccaggcc | ctgtggggag | gaagagctat | ccagctgtgg |
| 38101 | tgctgatgac | ttggggggac | ctatcttttg | gctcttaacc | taggggaggg | ggcagggtgc |
| 38161 | aggggagctg | tgacttggct | cttaacctgt | agggaggggg | caggggctgg | gggagctgtg |
| 38221 | acacacccca | gcttctgagt | cttggggtga | agacttaggg | gtaagtcacc | cttcccccag |
| 38281 | gcttctctat | tgactcgggg | aaaggtctgg | tgcgtgcaga | agagctgagc | tttgtggctg |
| 38341 | gagccccccg | cgccaaccac | aagggtgctg | tggtcatcct | gcgcaaggac | agcgccagtc |
| 38401 | gcctggtgcc | cgaggttatg | ctgtctgggg | agcgcctgac | ctccggcttt | ggctactcac |
| 38461 | tggctgtggc | tgacctcaac | agtgatgggt | gagtgggtag | agggccgtgc | cacctgaggg |
| 38521 | aggctgggtc | tagtagcccc | agtctggctg | aggccactta | gcctcctgct | ggctcctctg |
| 38581 | gccagggagg | acccacactg | aatgtttccc | tctctccata | gctggccaga | cctgatagtg |
| 38641 | ggtgccccct | acttctttga | gcgccaagaa | gagctggggg | gtgctgtgta | tgtgtacttg |
| 38701 | aaccaggggg | gtcactgggc | tgggatctcc | cctctccggc | tctgcggctc | ccctgactcc |
| 38761 | atgttcggga | tcagcctggc | tgtcctgggg | gacctcaacc | aagatggctt | tccaggtgtg |
| 38821 | acggggaact | ggaaaggctc | agggagggag | gggccacagg | agggatgggg | aagcccctca |
| 38881 | gaggtcaggg | tgtggtcttc | tgaggactca | gggagagagg | gtccctgagc | ttatgtctga |
| 38941 | gctgtaccat | ttaccagctt | tctgaccttg | gcaagttcct | aacctttttg | cgttagtaat |

### Bibliography

Amalfitano, A. and J. S. Chamberlain. (1996). The mdx-amplification-resistant mutation system assay, a simple and rapid polymerase chain reaction-based detection of the mdx allele. Muscle Nerve 19: 1549-1553.

Ascadi et al. (1996). Dystrophin expression in muscles of mdx mice after adenovirus-mediated in vivo gene transfer. Hum. Gene Ther. 7: 129-140.

Belkin, A. M. et al. (1996). Beta 1D integrin displaces the beta 1A isoform in striated muscles: localization at junctional structures and signaling potential in nonmuscle cells. J. Cell Biol. 132:211-216.

Belkin, A. M. et al. (1997). Muscle betalD integrin reinforces the cytoskeleton-matrix link: modulation of integrin adhesive function by alternative splicing. J. Cell Biol. 139:1583-1595.

Bertorini, T. E. et al. (1986). Muscle calcium and magnesium content in Duchenne muscular dystrophy. Neurology 32: 1088-1092.

Bradford, M. (1976). Anal. Biochem. 72: 248-254.

Bulfield, 0. et al. (1984). X chromosome-linked muscular dystrophy (mdx) in the mouse. Proc Natl. Acad. Sci. USA 81: 1189-1192.

Burkin, D. J. et al. (1998). A functional role for specific spliced variants of the α7β1 integrin in acetylcholine receptor clustering. J. Cell Biol. 143: 1067-1075.

Burkin D. J. and S. J. Kaufman. (1999). The α7β1 integrin in muscle development and disease. Cell Tiss. Res. 296:183-190.

Burkin, D. J. et al. (2000). Laminin and α7β1 integrin regulate agrin-induced clustering of acetylcholine receptors. J. Cell Sci. 113:2877-2886.

Campbell, K. P. (1995). Three muscular dystrophies: loss of cytoskeleton-extracellular matrix linkage. Cell 80:675-679.

Campeau, P. et al. (2001). Transfection of large plasmids in primary human myoblasts. Gene Therapy 8:1387-1394.

Collo, G. et al. (1993). A new isoform of the laminin receptor integrin alpha 7 beta 1 is developmentally regulated in skeletal muscle. J. Biol. Chem. 268: 19019-19024.

Colognato, H. et al. (1999). Laminin polymerization induces a receptor-cytoskeleton network. J. Cell Biol. 145: 619-631.

Conover, W. J. (1980). Practical Nonparametric Statistics. 2nd ed., John Wiley & Sons, New York.

Cordier et al. (2000). Mol. Ther. 1: 119-129.

Deconinck, A. E. et al. (1997a). Postsynaptic abnormalities at the neuromuscular junctions of utrophin-deficient mice. J. Cell Biol. 136:883-894.

Deconinck, A. E. et al. (1997b). Utrophin-dystrophin deficient mice as a model for Duchenne muscular dystrophy. Cell 90:717-727.

Denetclaw, W. F. Jr. et al. (1994). Myotubes from transgenic mdx mice expressing full-length dystrophin show normal calcium regulation. Mol. Biol. Cell. 5:1159-1167.

DiMario, J. X. et al. (1991). Fiber regeneration is not persistent in dystrophic (mdx) mouse skeletal muscle. Dev. Biol. 148:314-321.

Donoviel, D. B. et al. (1996). Analysis of muscle creatine kinase gene regulatory elements in skeletal and cardiac muscles of transgenic mice. Mol. Cell. Biol. 16:1649-1658.

Ebihara et al. (2000). Differential effects of dystrophin and utrophin gene transfer in immunocompetent muscular dystrophy (mdx) mice. Physiol. Genomics 3:133-144.

Fujii et al. (2000). Targeted and stable gene delivery into muscle cells by a two-step transfer. Biochem. Biophys. Res. Commun. 275:931-935.

Gilbert et al. (1999). Efficient utrophin expression following adenovirus gene transfer in dystrophic muscles. Biochem. Biophys. Res. Commun. 242:244-247.

Grady, R. M. et al. (1997a). Subtle neuromuscular defects in utrophin-deficient mice. J. Cell Biol. 136:871-882.

Grady, R. M. et al. (1997b). Skeletal and cardiac myopathies in mice lacking utrophin and dystrophin: a model for Duchenne muscular dystrophy. Cell 90:72-738.

Grady, R. M. et al. (1999). Role for alpha-dystrobrevin in the pathogenesis of dystrophin-dependent muscular dystrophies. Nat. Cell Biol. 1:215-220.

Greelish et al. (1999). Nat. Med. 5:4439-443. Xiao et al. (2000), Cordier et al. (2000).

Hayashi, Y. K. et al. (1993). Abnormal localization of laminin subunits in muscular dystrophies. J. Neurol. Sci. 119:53-64.

Hayashi, Y. K. et al. (1998). Mutations in the integrin α7 gene cause congenital myopathy. Nat. Gen. 19: 94-97.

Hodges, B. L. and S. J. Kaufman. (1996). Developmental regulation and functional significance of alternative splicing of NCAM and α7β1 integrin in skeletal muscle. Basic Appl. Myology 6:437-446.

Hodges, B. L. et al. (1997). Altered expression of the α7β1 integrin in human and murine muscular dystrophies. J. Cell Sci. 110: 2873-2881.

Hynes, R. O. (1992). Integrins: versatility, modulation, and signaling in cell adhesion. Cell 69:11-25.

Jaynes, J. B. et al. (1986). Transcriptional regulation of the muscle creatine kinase gene and related expression in transfected mouse myoblasts. Mol. Cell. Biol. 6:2855-2864.

Johnson, J. E. et al. (1989). Muscle creatine kinase sequence elements regulating skeletal and cardiac muscle expression in transgenic mice. Mol. Cell Biol. 9:3393-3399.

Kaplan, E. L. and P. Meier. (1958). Nonparametric estimation from incomplete observations. J. Am. Stat. Assoc. 53:457-481.

Kim, Y. Y.et al. (1999). Cellular localization of α3β1 integrin isoforms in association with myofibrillogenesis during cardiac myocyte development in culture. Cell Adhesion and Comm. 7:85-97.

Kwon, M. S. et al. (2000). Calreticulin couples calcium release and calcium influx in α7β1 integrin-mediated calcium signaling. Mol. Cell Biol. 11:1433-1443.

Law, D. J. et al. (1994). Talin, vinculin and DRP (utrophin) concentrations are increased at the mdx myotendinous junctions following onset of necrosis. J. Cell Sci. 107:1477-1483.

Leung, E. et al. (1998). A novel extracellular domain variant of the human integrin α7 subunit generated by alternative intron splicing. Biochem. Biophys. Res. Commun. 243: 317-325.

Lim, L. E. and K. P. Campbell. (1998). The sarcoglycan complex in limb-girdle muscular dystrophy. Curr. Opin. Neurol. 11:443-452.

Martin, P. T. et al. (1996). Synaptic integrins: selective association of the al and α7A, and α7B subunits with the neuromuscular junction. Dev. Biol.174:125-139.

Matsuda, R. et al. (1983). Regenerating adult chicken skeletal muscle and satellite cells cultures express embryonic patterns of myosin and tropomyosin isoforms. Dev. Biol. 100: 478-488.

Matsumura, K. et al. (1992). Association of dystrophin-related protein with dystrophin-associated proteins in mdx mouse muscle. Nature 360: 588-591.

Matsumura, K. and K. P Campbell. (1994). Dystrophin-glycoprotein complex: its role in the molecular pathogenesis of muscular dystrophies. Muscle Nerve 17:2-15.

Mayer, U. et al. (1997). Absence of integrin alpha 7 causes a novel form of muscular dystrophy. Nat Genet. 17:318-323.

Monaco, A. P. et al. (1986). Isolation of candidate cDNAs for portions of the Duchenne muscular dystrophy gene. Nature 323: 646-650.

Muzyczka, N. (1992). Curr. Top. Microbiol. Immunol. 158:97-129.

Oda, T. et al. (1993). Longitudinal study of spinal deformity in Duchenne muscular dystrophy. J. Pediatr. Orthop. 13:478-488.

Peto, R. et al. (1977). Design and analysis of randomized clinical trials requiring prolonged observation of each patient. II. Analysis and examples. Brit. J. Cancer35:1-39.

Poirier et al. (2000). Increased intracellular triglyceride in C(2)C(12) muscle cells transfected with a human lipoprotein lipase. Biochem. Biophys. Res. Commun. 270:997-1001.

Pons, F. et al. (1994). Does utrophin expression in muscles of mdx mice during postnatal development functionally compensate for dystrophin deficiency. J. Neurol. Sci. 122:162-170.

Rafael, J. A. et al. (1998). Skeletal muscle-specific expression of a utrophin transgene rescues utrophin-dystrophin deficient mice. Nat. Gen. 19:79-82.

Rafael, J. A. et al. (2000). Dystrophin and utrophin influence fiber type composition and post-synaptic membrane structure. Hum. Mol. Genet. 9:1357-1367.

Ragot et al. (1993) Nature 361: 647.

Sand, A. D. et al. (1987). Immunochemical analysis of protein isoforms in thick myofilaments of regenerating skeletal muscle. Dev. Biol. 119:336-349.

Saher, G., and E. Hildt. (1999). Activation of c-Raf-1 kinase signal transduction pathway in alpha(7) integrin-deficient mice. J. Biol. Chem. 274:27651-27657.

Shield, M. A. et al. (1996). E-box sites and a proximal regulatory region of the muscle creatine kinase gene differentially regulate expression in diverse skeletal muscles and cardiac muscle of transgenic mice. Mol. Cell. Biol. 16: 5058-5068.

Sicinski, P. et al. (1989). The molecular basis of muscular dystrophy in the mdx mouse: A point mutation. Science 244:1578-1580.

Song W. K. et al. (1992). H36-α7 is a novel integrin alpha chain that is developmentally regulated during skeletal myogenesis. J. Cell Biol. 117:643-667.

Song, W. K. et al. (1993). Expression of α7 integrin cytoplasmic domains during skeletal muscle development: alternate forms, conformational change, and homologies with serine/threonine kinases and tyrosine phosphatases. J. Cell Sci. 106:1139-1152.

Stedman, H. (2001). Molecular approaches to therapy for Duchenne and limb girdle muscular dystrophy. Curr. Opin. Molec. Therap. 3:350-356.

Tinsley, J. M. et al. (1996). Amelioration of the dystrophin phenotype of mdx mice using a truncated utrophin transgene. Nature 384:349-353.

Turner, P. R. et al. (1988). Increased protein degradation results from elevated free calcium levels found in muscle from mdx mice. Nature 335:735-738.

Vignier, N. et al. (1999). Structure, genetic localization, and identification of the cardiac and skeletal muscle transcripts of the human integrin alpha 7 gene (1TGA). Biochem. Biophys. Res. Commun. 260:357-364.

Van der Flier, A. et al. (1995). A novel beta 1 integrin isoform produced by alternative splicing: unique expression in cardiac and skeletal muscle. FEBS Lett. 369:340-344.

Von der Mark, H. J. et al. (1991). Skeletal myoblasts utilize a novel al-series integrin and not α6β1 for binding to the E8 and T8 fragments of laminin. J. Biol. Chem. 266:23593-23601.

Wang, W. et al. (1995). Localization of the α7 integrin gene (ITGA7) on human chromosome 12q13: Clustering of integrin and hox genes implies parallel evolution of these gene families. Genomics 26:563-570.

Wang, W. et al. (2000). Adeno-associated virus vector carrying human minidystrophin genes effectively ameliorates muscular dystrophy in mdx mouse model. Proc. Natl. Acad. Sci. USA 97:13714-13719.

Xiao et al. (2000). J. Virol. 72:10222-10226.

Yoon and Lee. (2000). Nature 408:483-488.

Zhidkova, N. J. et al. (1995). Novel isoform of beta 1 integrin expressed in skeletal and cardiac muscle. Biochem. Biophys. Res. Commun. 214:279-285.

Ziober B. L. et al. (1993). Alternative extracellular and cytoplasmic domains of the integrin α7 subunit are differentially expressed during development. J. Biol. Chem. 268:26773-26783.

Ziober B. L. et al. (1996) Identification and characterization o f the cell type-specific and developmentally regulated α7 integrin gene promoter. J. Biol. Chem. 271:22915-22922.

1. A recombinant cell containing recombined within its genome a reporter gene construct comprising a transcription regulatory sequence of a human α7 integrin gene and a reporter coding sequence, wherein said transcription regulatory sequence is operably linked to said reporter coding sequence.

2. The recombinant cell of claim 1, wherein said cell is a cultured muscle cell or a myoblast.

3. The recombinant cell of claim 2, wherein the reporter coding sequence is selected from the group consisting of a green fluorescent protein, yellow fluorescent protein, luciferase, β-lactamase, β-galactosidase, chloramphenicol acetyltransferase or β-glucuronidase, and an immunological tag portion coding sequence.

4. The recombinant cell of claim 2, wherein said cell is a human cell or a mouse cell.

5. A method for identifying a small molecule composition which increases expression of an α7 integrin gene, said method comprising the steps of:
(a) contacting the recombinant cell of claim 2 with a small molecule test composition to produce a contacted recombinant cell;
(b) monitoring reporter coding expression in the contacted recombinant cell and monitoring expression of the reporter coding sequence of the reporter gene construct in a recombinant cell which has not been contacted with the small molecule test composition;
(c) determining that the small molecule test composition increases reporter coding sequence expression when the expression of the reporter coding sequence is greater in the contacted recombinant cell than in the recombinant cell which has not been contacted with the small molecule test composition,
whereby a small molecule composition is identified which increases the expression of an α7 integrin gene when the expression of the reporter coding sequence is greater in the contacted recombinant cell than in the recombinant cell which has not been contacted with the small molecule test composition.

6. The method of claim 5, wherein the monitoring and determining steps are carried out in a high throughput assay format.

7. A method of alleviating symptoms of a muscular dystrophy which is characterized by levels of α7 integrin which are lower in a patient suffering from or susceptible to said muscular dystrophy than in a normal individual, said method comprising the step of administering to a patient suffering from or susceptible to the muscular dystrophy a composition identified by the method of claim 5.

8. The method of claim 7, wherein said muscular dystrophy is Duchenne muscular dystrophy.

9. A method for alleviating symptoms of a muscular dystrophy which is characterized by levels of α7 integrin, dystrophin and/or utrophin which are lower in a patient suffering from or susceptible to said muscular dystrophy than in a normal individual, said method comprising the step of administering to a patient suffering from or susceptible to the muscular dystrophy a DNA construct comprising an α7 integrin coding sequence operably linked to a transcription regulatory sequence which enables selective expression in muscle cells and a vector sequence.

10. The method of claim 9, wherein the vector sequence is a virus vector sequence or a plasmid sequence.

11. The method of claim 9, wherein the step of administering is by intravenous administration.

12. The method of claim 9, wherein the step of administering is by intramuscular administration.

13. The method of claim 9, wherein the step of administering is by regional perfusion.

14. The method of claim 9 wherein the muscular dystrophy is Duchenne muscular dystrophy.

15. The method of claim 9, wherein the step of administering comprises ex vivo transformation of stem cells or myoblasts isolated from the patient to produce transformed myoblasts and subsequent administration of the transformed stem cells or transformed myoblasts to the patient with the result that the transformed myoblasts differentiate to form muscle cells which express α7 integrin in the patient, whereby the symptoms of muscular dystrophy are ameliorated.


SEQUENCE LISTING (11 sequences)

SEQ ID NO: 1 - DNA, 20 bp, Artificial
  Oligonucleotide useful as a primer
caagctgcacgcctgggtcc

SEQ ID NO: 2 - DNA, 25 bp, Artificial
  oligonucleotide useful as a primer
ggcacccatgacgtccagattgaag

SEQ ID NO: 3 - DNA, 27 bp, Artificial
  oligonucleotide useful as a primer
catagttattaatgcatagatattcag

SEQ ID NO: 4 - DNA, 20 bp, Artificial
  oligonucleotide useful as a primer
gaacagcacctttctggagg

SEQ ID NO: 5 - DNA, 20 bp, Artificial
  oligonucleotide useful as a primer
ccttgaactgctgtcggtct

SEQ ID NO: 6 - DNA, 1970 bp, Homo sapiens
gaaagtagaatcctggtgccagccctgctgacagcatatgtatttccttatagtacctgtttagagatgtgttagtgctctggaggggatagccacaggtgtagtattggaaaacagagggccagacttccaaatgtctgttaacttatccaaggcaaagactgtcccagggcagcagagtaagaacccactttttttttgttttcaaagaagtataatcctgaacaatgaagtaggaaagacagaacacaggaagaggaaggaggtaggacacttattggaacttttaagaaagggaaagagaagaaagaatcgtaagaatatgatagtgtttgaagggcagagacaacactagaaacattgagaaatactctgagaaagattccaagtgtggcagagacaagaatgatgacaaaatagaatttgggatgagacaaaatcagatagtgagagagagaagggaagatggacagatgtatattcacaagaccaacaccagtaagcaaggggagtaggaaggggaagtgggagcattcgaggttcccattatgccaaattatttcctgtctctccttctggccccatttctgtatcggagttataaatagcagagagttggaaagtgtccccccacccccttgcctctgtcccagcctgagggaaagggagaggaagagggacaggccaatgggtccctgtggagatcccatctcagccccacccaggtcctgctgagccagtccaggactctgccccctcccatcccctttcatggataggaaatgtgcagtcctgggacgggtctggtagctggggacaccctttacatccctctgcctcttgggtccagtctctttcatctttgccttctttgacacccactcccctccccactgcttaatttcctcttcctgtaatcatccccagtcgttttcttttctcccttcattccatcccttgtcaattaatctcttgcccttctttcttcctctctattcctttcctttttccatttctccatttgctccccgtatctcccgagtttctctctctcttcttgcctctttttctctgttcccttgaatcctgacgatgtggctagcactgctgtggtcattgccgggctgggggcgggggatgggataggatgggggagggcagcggtctgatcccaacagcagaaagagtgctctatgtgaccatgggggaacagggagcactaagatgccacgctgcacccaggcccaggacggctcccctttcatttcctctctatctgcacatctctcttcccaggttgtcttttagcgtcttcccaacttctcatctcttaccctccttcctctgtttcagcccctctctttctatctgtacttctctccctccgcattccaaggcgccgcctccaccactcccggggtggggatggggttgggggagaaggggaggagagcgccgcgcaggggcggagccggagacggtgctgggcttggggggcgtggtggtggggggtcagcaaggctagtttccatcccagccaccagcctgggcatccccttggagacgggcttgggtctccacctgccgcgggagcgaggggcggggccggaggcggggcctgagtggcgtccccgggagaggaggcgggagccggagtgggcgccggagctgcggctgctgtagttgtcctagccggtgctggggcggcggggtggcggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggct

SEQ ID NO: 7 - DNA, 20 bp, Artificial
  oligonucleotide useful as a primer
caagctgcacgcctgggtcc

SEQ ID NO: 8 - DNA, 35 bp, Artificial
  oligonucleotide useful as a primer
aatatttagcggccgcgggcatcggtcgacggatc

SEQ ID NO: 9 - DNA, 31 bp, Artificial
  oligonucleotide useful as a primer
tatctcgagcttatatccctggtgtctagcc

SEQ ID NO: 10 - DNA, 39 bp, Artificial
  oligonucleotide useful as a primer
tatctcgagttaattaatctcattccacctgaatcttcc

SEQ ID NO: 11 - DNA, 13980 bp, Homo sapiens
ccactgaattccctcaatcacatttatgttcttttcctcccagccactcccatggttcaagctttgactacaaccagaattcagaggcaggcagaaggattccagtgctggagaggtgagtgaagtaaaaaagttctcatggtgtgcatgttgggacggaaaagcctgaccttgggacataagctccaaggctctgttgccagatgaggtggagggagaagttagccctgaagtgtgtgttctggaagtgtttgcttgtaagctagagacaacagttgcaaaaagtgtgatttgagggagctgaaaaatactgatctcaaagtggggaagaagatgttgaaaagggaaggagctggagaaagcctcagcttccactcatacaaaagctaaagggctaaaatcttggctggatctggacatttctcaacgtctaaaattttggaaatttttataaagattattaatctttcatttttacatttaatttatttaaaaagttcagtttctcagtcatactagccacatttctttttctttttctttttttttgagacagagtctcactctgttgcccaggctgaagtacagtgtattaatctattccatggagtggagtggataatctattccatggattatcattttactttgttagtggtatccttagaagcacaaaatttttaaatttttttttttttttgaggcagagtctcactgtgtcccccaggctggagtgcagtggtgctatctctgctcactgcaggctccgccttctgggttcaagcgattcttgtgcctcagcctcctgagtagctgggattacaggtgtgtaccaccacgcccagctgatttttgtatttttagtagagatgggtttttgtcatattggccaggctggtctcaaactcctgacctcaagtgatccacctccctccctcccaaagtgctgggattaccatgtctggcttggaaattattttgaaataattatagatcagaggaagttgtaaaaatagcacatgaagtcttgtgtacctttcactcagtttcccctaatggtgacatcttatgtaactgtagcataaaatcaaaaccaaaaagttgatattggtacagtattgttaactggcctgcagacctcactcagttttcaccattttttacatgcatttatttgtttgtttgtagttctgtgcagttttatatcttgtatagatttggataatcaccaccacaatcaagatacaaaacccatcaccacaaaggaacccccttgtgctattcctttatgtttgtccccacccccctccatccttgtcccctggcagccagtaatctggtcttcatttctatagttttgtcattttgagaatggtatgcgagtggaataatacagtttcagcattttttgtttggagacagggtctcactctatcacccaggctggagtgcagtggcaagatcatggctcactgcagccttcacctcctgggctcaagtgacactcccgcctagcctcctgagtagctgggaccacagatttggctaacttttctattttttgtagagatgggggtctccctatgttgcccaggctggtctccaactcctgggctcaagtgatcctcctgccttggcctctcaaagtgctgggattgcaggcatgagccactgtgcccagctcagcattaatttttaatttaactaattcctaagctcttgactgaaatacaagaagttctctaacagtttatttattttaatattgagcttaccgcattctctggatccttctagtttcttttttttttctttttttctgatgtggagtctctgtcacccaggctggagtgcagtggtgccatctcagctcactgcaacctccgtctcctgggtttaagtgattcttgtgcctcagtctctggagtagctgggattacaggtacccgccaccacacccggctaatttttgtatttttagtagagacagggtttcaccgtgttggtccggctggtcttgaacttctgatctcaggtgatccgcctgcttcggcctcccaaagtgctgggattataggcgtgagccaccgcgcccggccccgtctagtttcttaatttccctcttcacctacgatattatcttccactccaacattctggtctcatttctccttgagagaaatctacatgtctaaatttactaggctggtctagcacgctcttgtgtgttcccctccctcctttgcccctctatttatagccaggctaattttgggtggcctctctctctcttctttcctgatctttcctcctgtggtggtgaggtgacttctcaaatatttggagagaggaggtcagaagcagattcttggcatctgatttcagccctggatcacagaagccagtggagtgggaatggagacaggcagaagctgcaggtgcagataggaggcagcttgggctctaaaggcattttgagctgggtcgggggcggggggacctgggcagggagtcagtagtcccagttctgtcctaattttgcaattctgcattcccatgtcagctcttctctactgtctggggctctgagatattaaaaaggatggggagggcatggtgaaagtagaatcctggtgccagccctgctgacagcatatgtatttccttatagtacctgtttagagatgtgttagtgctctggaggggatagccacaggtgtagtattggaaaacagagggccagacttccaaatgtctgttaacttatccaaggcaaagactgtcccagggcagcagagtaagaacccactttttttttgttttcaaagaagtataatcctgaacaatgaagtaggaaagacagaacacaggaagaggaaggaggtaggacacttattggaacttttaagaaagggaaagagaagaaagaatcgtaagaatatgatagtgtttgaagggcagagacaacactagaaacattgagaaatactctgagaaagattccaagtgtggcagagacaagaatgatgacaaaatagaatttgggatgagacaaaatcagatagtgagagagagaagggaagatggacagatgtatattcacaagaccaacaccagtaagcaaggggagtaggaaggggaagtgggagcattcgaggttcccattatgccaaattatttcctgtctctccttctggccccatttctgtatcggagttataaatagcagagagttggaaagtgtccccccacccccttgcctctgtcccagcctgagggaaagggagaggaagagggacaggccaatgggtccctgtggagatcccatctcagccccacccaggtcctgctgagccagtccaggactctgccccctcccatcccctttcatggataggaaatgtgcagtcctgggacgggtctggtagctggggacaccctttacatccctctgcctcttgggtccagtctctttcatctttgccttctttgacacccactcccctccccactgcttaatttcctcttcctgtaatcatccccagtcgttttcttttctcccttcattccatcccttgtcaattaatctcttgcccttctttcttcctctctattcctttcctttttccatttctccatttgctccccgtatctcccgagtttctctctctcttcttgcctctttttctctgttcccttgaatcctgacgatgtggctagcactgctgtggtcattgccgggctgggggcgggggatgggataggatgggggagggcagcggtctgatcccaacagcagaaagagtgctctatgtgaccatgggggaacagggagcactaagatgccacgctgcacccaggcccaggacggctcccctttcatttcctctctatctgcacatctctcttcccaggttgtcttttagcgtcttcccaacttctcatctcttaccctccttcctctgtttcagcccctctctttctatctgtacttctctccctccgcattccaaggcgccgcctccaccactcccggggtggggatggggttgggggagaaggggaggagagcgccgcgcaggggcggagccggagacggtgctgggcttggggggcgtggtggtggggggtcagcaaggctagtttccatcccagccaccagcctgggcatccccttggagacgggcttgggtctccacctgccgcgggagcgaggggcggggccggaggcggggcctgagtggcgtccccgggagaggaggcgggagccggagtgggcgccggagctgcggctgctgtagttgtcctagccggtgctggggcggcggggtggcggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggtgagtcaccgcacccgcccagagtcgccatgcccgagccacagatcgtccccctccccactctgtgggcctcctcatttctctgttttctagccccaccaagacctagactgcccacagacatcccacatcccaacctggagccttgcctcatctggcttgcgtctgaagctgcacttcccggccctgagaccagtattttgctttagggatgagttggaaagcaaggttcttgtcttggcagcgaaccatctccttcttctgggcctttcccccaacttgcatccttgatccagccccagggcctctggctcccctgcttcttccaagggctgaattccccaagggagggagactgtctgtctctgcttagaatgggaggagatggaaaggacatagaagttgagggtgccatgagagggatgcatgcagggcagactccagaaataacttcctgctagagcattgccatggatggaatgagggcagcagggcactggaaggccaggagagagcttccacttctgtggcttaagaccacgggaagattgggagaggatctgcaggtctgccaacctgcagtaggtggcttggtgatagagagtggcagcaaactgaaccctcaaagtactagtagcagtagtagtagccgcagcagctgtagcagtgagagagatccaggaaggatgctggccaggctgctccccttcctcctccttagcaaatttccaactccaggaatctcagcagctgggaagggccaggaggagtaaggggtggaggacaattctaattttttctaatcagttcaggacccatgggagatggatatacttttgtgaggggcctgtgactggtcatgttgcctgtatccttggctcttgctacatgtctgattgtaaaaagggaggccagaggtgaagaaagcttctcacctgctcctgctagggggcttttctctcttcaaccagtgcctaagccacattaagtatccattactgggatcaatgctgtccactgggactgtcttctgcctctactgtcggtctgggggcagggggcagggacaagagctcatttctcctcacttgcttggggagtgggggcctagctctaatctttcttcttccattatccctatcatctggtagcagggtcggtggtccccaaaactttgggagagatagaaagcaacggacttcatctcctcttctgtttaccatctgcttcctcattcacctttgctccctccctcccttcctccctccttctccatctgtcagagttcgaggactggaggcctttttaggacatgctgaactctctaagctatttccaggcaaattctaggttatttttaatagcttggtctcttgtcatttccccctcctctctgaaggtggcccctggttccgtctcccagagccaagctggggcctttcccagagggcctgactgcctcaccctgcttttgctccagcagggggtgctctgctgctggggggcggggggtatgtgagaggccaggcacctgctcagtccctagcttttgagttgcaggtggcctgccttagcactcactgatgaaaaaaacttcttgcctgttttgatgtcttttagtctagctctgggatgagactttaaggtctaacctttgctgtgtggttccagcctcatttacttccctcaactgtaaaaaggatataaacatagtattactacatagggttgttgtgcagattaagagttcttaatatatataaaatgcttagaatagtgcatagcccgcagtgagtgctgtgaagtgttagtagtattgctattcttgtattgtgattcacagagcgccttacagagattctggatccaaaggcttggctagagggcctccctggctgagccagccttccaggccaagcatcctccccagagggccacccagattgagaggggccaaagaggggctggacttgggctggggccctggagtgtgtggagaatcgagaagtgcagtggtcgtgggctactcctttgtcttcacttagctgagctcccagggggtccctctgccccccagctgccaacacttttttttttttttttttgcttttctctctgcagtggctacactgtggctgtccagaagactggggtggttagggcgtatggcatgaagccaggaaggagtgtgtgtggctggaccagaggtggagggactagagaggatgctgctgggtgctcttgttccactaaggatcgattggtctcttctccaccaagagcggactgggcatatctatgcactcagcttctttcttccacatgggcccctcccctcctccctacttttggcctccagaggagatgtgaacatagaacaaggataacttatctgggtgcttagctatgcactgaccagctgtgacactgggtatctctatgagtccacaaaatgtgtgtgttcagtaaacacattctgacactccctatggggcaagcacaaagatgaaaagacagccccgacactcagagagatggccacttctatgtttggaggctgggggtactgctgacttgcctgaaggttgccatttatttatgcagggctgtatcaccccgtttccttttctgcccagggtaccctcatctccccactctctccttccctttctggggtggtctcagtgttctagagacaggtcagtcactgggtggagtgacaaagtgttggagttaggcccatgtggatttgaattccagcatcactgcttaatgtctttgagtgagttttctcatctgaaagacaagaaaagaatccttatctcataggattgttctgatgattaaatgacataatgcatgtgacttgcctatcctggtgcttggcacatatgtggacagtgatgaatgttagtttcttatatccctggtgtctagcctcggatctgacgtcatagtaggtgctgaataaatatgatttccttgtctcaccagcgcctgacacagggcttggcatacaatagactctcaataagtagttgaatgccaaatgtgtcttctcttctctactactccctataccccttctctgtcttgacactggctctgacaagggatggcagctgctaagagatgaggaggagttgtgggaaggaagaatggctctctgccctccccctccaccccatcagagctggcacagtgccccacagatgcctgtctgtaatactgcctaacatggttttgggccttgccccccaggaagggagatggaggagaagagtgtgggagagaggcgttgaggtttgtcccactgccacttctgagtctctccttctgcaaagagaggacccatggagccagctgggtgtcagtcatcttacctcacccccgccttcactctggcttgggggttcagccccaggggacccaggcagcctccattcccagcactgtgctcccctggggaagacggcttggctgtgatcatggaaaattgtcctgccaagaaagttgtagctgggaaagaggctgagggggaggcaggagagaagactgggtgggggtggaagggaaggagaaatcatggacatggggagaaggaaggatggggaaggggattcaggatgtcgggaagagaatggggtagcattggaggcagaaggagaaacttgtccctacctccatgccagccagagtgactgatggaatcctgggctggcacagcttctgggaggtggggtctttgctgggtccctgatgagggggcagtgggtccgtatctagcctcttgcctggcctctgaagctggtccctgagccacactctgatgccagtctggggccctgttacttttgctcccagcattcttggcatttctggctgggtttcaactggactgggttggggagcagggcagagcttggggatggggccaaggaggggatagggaaggcctactcaggaacaggtgctgggaacaggcagttctttcaaaccagcactgttggcctggctgcttgggttggcgtgtatgtgtgtgtgtgtgtgtgtgtgtgtatctactgtgtatgttgatcccttatccagatagtatgtacatgcaacgtgatgactgcatgaccaagcatattaatttgtccttgccagggtttgagaaaactgacatttgccccttctctttagtccttgaacactctctttagtactgaggggttgggcctgggcagctctaatgagattgggtcattctgacctctaactcctgtccctgtccctgcccctgccccatcttgcaggctgctggtgggtgctccccaggccctggctcttcctgggcagcaggcgaatcgcactggaggcctcttcgcttgcccgttgagcctggaggagactgactgctacagagtggacatcgaccagggaggtgtggccctgcatgaacagagtgggggaagcgtgtgagcggggaggagaggacttgggctcctcttccctcccctaattcccagtgtcctgcctctagctgatatgcaaaaggaaagcaaggagaaccagtggttgggagtcagtgttcggagccaggggcctgggggcaagattgttgtgagtattgcttctcatgactgaatgcacggatggggtgtgtgtgtgtgtgtgtttatggtgtgtgcatacgcataggtgtgcttagagaacacaagttaggaatatggtatgattccaagtacatcagggagatataaaaaggtgtgagacatggtccttgtccttataaatgtaaaaatgtctgtccattcattcatccatccatttgtcaaactcttactgagaaccttttaagcatcaggcattgtgctagttactacaggggaaggctcatgcctgtaatcccagcactttcggaggccgaggcaggtggatcacctgaggtcaggagttcgagaccagccggaccaacatggcgaaaccctgtctctcctaaaaatacaaaaaaattagccgggcgtggccgggtgcggtggctcacgcttgtaatcccagcactttgggaggccgaggtgggtggatcacgaggtaaggagatcgagaccatggtgaaaccccgtctctactaaaaacacaaaaaattagccgggcgtggtggcgggcgcctgtagtcccagctactcagagaggctgaggcaggagaatggcgtgaaccggagaggcggagcttgcagtgagctgagatcgcgccactgcgctccagcctgggtgacagagcgagactccgtctcaaaaaaaaaaaaaaattagctaggtgtggtggcaggcgcctgtaatcccaggtactcgggaggctgaggtaggagaatcacttgaacctgggtggaggaggttgcagtgagctgagatcgcaccattgtaccctagcctgggagacaagagcaaagttccgtctcaaaaccaaccaaacaaacaaacgaaaaaaccagagctctctgtttctctctctctctctatctttcagtaacacgcatagatacacaattaccaatacagatcactgtggggcagaatctggttcatgttaagtgagtggtctagtctccagtctataaaagtccaaaggaggagtagagagaagacttctgcagaggggatgatttgagccaggctttaataataggtaatacctagcctgtgcaacatagtgggacctcatctttataaaaaataaaaacaaattagccagtcatggtggtgcatgcctgtagtcccagctacacaggaggctgaggtgggaggatcacttgagccctggaggtcgaggctgcagtgagccatgattgtgccactgcactccagcctgggtgacagagtgagaccttgcttcaaaaaaaaaaaaaaagtaatacttggagagtgaagcggacaggaagttctttgcagatgagatggtgacacttacaaaggtccagggacagggccaagcttggcattttggaggactgtgacatgatcagggagacacacatcctatgtggtggcttaattgtgtcttttggctccaggcagaatgtggaacaaggagatctccatttgagggcaaggaagtgggtgcagacaggttgctgggttatgcatggacctgtgtaacactggcagggtaatggtgcttgagtggtgccggcataggggtgtgtgtgtatgtgtgcatgtgcatgtgcatgtgagcacacatgtatcagtatctgccaaatctctgcatatgggcagcatgcctcaagcaggtccctggcccacagagtgaaatgatccccatcccttcctcccccagacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaagggtgagtcactcctcgggaaggggagaaggggaccaaaacctcctcttacctcagagacagggttggggatggcacatggccaagcatgaccacatgtgcactgctgtatggccccagggcactgccatgccttccaccccattgagctagtgcacacatgaatggggggtgcctcctttccctcgcacggccaagtgttcctcaacatgctggcatgggccccaagtgcacgctgggcctgcagctggggcctgcatgctccaacacactagcccacacctcatcactgccattcccgtctccgcacgctgctgctggctgagctgacactcggtgagtgtgatgccacatctgggggaccccaggaagcctgggttggggacagggtggggagagggctagaaagaagaggcagggcttccccgtgtgcctgtctaactcagtgtccggcctgaggggtgttccttgcgccctgccctgggcactaacaggtctgtccttgcaggcacggccagggtggagctctgtgcacagggctcagcggacctggcacacctggacgacggtccctacgaggcggggggagagaaggagcaggacccccgcctcatcccggtccctgccaacagctactttggtagggacctctccccggcccagaactgctctaaccctctgctcctctctcttgtcctctctctccatgctcccatccttctgtctctgtttctgtctctcaccttgtctctctctgtctttctgtctctggctgtgatctctctggtctctttttctctctccacctcttcttcttccaccattttctggcctttctgtggctctgtctccctactctgtggcccctactctggatgtcccctccctggtgtctcaccccaccccccacagggttgctttttgtgaccaacattgatagctcagaccccgaccagctggtgtataaaactttggaccctgctgaccggctcccaggaccagccggagacttggccctcaatagctacttaggtttgtaagctcccacctcctggactctaggggcatggcccagcctcccctccttccccagggaactcgacctttggtgccttataatctcctcctcccccaacacacacccagggagacatacattgggcccaaattgcagagaagagctgggtccaatgatcaggcctaagaggaggaggcccccagggtggtggcctctggggctgtgagccaggggtctccatggaggaagattcaggtggaatgagagggccagggctgaggatattttgggaaggacagtcctgtcttctagggggactttccctgaggggatggatggtgggcacatattgaagaaagggctaatgttgttggtaagtccctctcgttgtctcatctgcattcctctgcagaggaggaggaaaccaggcctgggagatgtttgggtgaagcaggcgctctctcactcccccttgtctccccctcatccatgtgaagacttcccctccctgccaggatgagggagttgggggaaagaggtgcactgggtgggattcgggcctgagagggacctctagctcttctagctccctgggtgtgggcagggtgaggccactgtgctcagcctcctacctgggctcctggccttctcagccatcacctttctctctcttgcccagtccctgaggctgacctcactgcactttttgtgccaagcttgtctctgggcctggtgggtgtgggaggctgccaggccctgtggggaggaagagctatccagctgtggtgctgatgacttggggggacctatcttttggctcttaacctaggggagggggcagggtgcaggggagctgtgacttggctcttaacctgtagggagggggcaggggctgggggagctgtgacacaccccagcttctgagtcttggggtgaagacttaggggtaagtcacccttcccccaggcttctctattgactcggggaaaggtctggtgcgtgcagaagagctgagctttgtggctggagccccccgcgccaaccacaagggtgctgtggtcatcctgcgcaaggacagcgccagtcgcctggtgcccgaggttatgctgtctggggagcgcctgacctccggctttggctactcactggctgtggctgacctcaacagtgatgggtgagtgggtagagggccgtgccacctgagggaggctgggtctagtagccccagtctggctgaggccacttagcctcctgctggctcctctggccagggaggacccacactgaatgtttccctctctccatagctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccaggtgtgacggggaactggaaaggctcagggagggaggggccacaggagggatggggaagcccctcagaggtcagggtgtggtcttctgaggactcagggagagagggtccctgagcttatgtctgagctgtaccatttaccagctttctgaccttggcaagttcctaacctttttgcgttagtaat